(19) United States
(12) Patent Application Publication
(10) Pub. No.: US 2024/0392015 A1
(43) Pub. Date: Nov. 28, 2024

(54) LONG-ACTING ANTI-CCR5 BINDING AGENTS FOR THE PREVENTION AND TREATMENT OF HIV

(71) Applicants: Oregon Health & Science University, Portland, OR (US); CytoDyn, Inc., Vancouver, WA (US); University of Massachusetts, Boston, MA (US)

(72) Inventors: Jonah Sacha, Portland, OR (US); Scott Kelly, Vancouver, WA (US); Helen Wu, Portland, OR (US); Gabriela Webb, Portland, OR (US); Diogo Magnani, Boston, MA (US); Kathleen Engelman, Boston, MA (US)

(21) Appl. No.: 18/527,061

(22) Filed: Dec. 1, 2023

Related U.S. Application Data
(60) Provisional application No. 63/429,917, filed on Dec. 2, 2022.

(51) Int. Cl. C07K 16/28; A61K 39/00; A61P 31/18

(52) U.S. Cl. CPC C07K 16/2866; A61P 31/18; A61K 2039/505; A61K 2039/545

(57) ABSTRACT

The present disclosure provides CCR5 binding agents with increased effector function and circulation half-life that are useful for preventing or treating HIV.

### PRIORITY CLAIM

The present application claims priority to U.S. Provisional Patent Application Ser. No. 63/429,917 filed Dec. 2, 2022 and titled “LONG-ACTING ANTI-CCR5 BINDING AGENTS FOR THE PREVENTION AND TREATMENT OF HIV,” which is incorporated by reference herein in its entirety.

### GOVERNMENT SUPPORT

This invention was made with government support under grant no. 1R01AI154559-01 awarded by The National Institutes of Health. The government has certain rights in the invention.

### REFERENCE TO AN ELECTRONIC SEQUENCE LISTING

The contents of the electronic sequence listing (438_SeqListing.xml; Size: 26,918 bytes; and Date of Creation: Aug. 16, 2024) is herein incorporated by reference in its entirety.

### TECHNICAL FIELD

The present disclosure provides CCR5 binding agents with increased effector function and circulation half-life that are useful for preventing and treating HIV and methods of use thereof.

### BACKGROUND

Currently, the most people ever in history are living with HIV due to continued new infections and greater access to antiretroviral treatment. To effectively slow the epidemic, approaches are needed to halt new infections and to suppress viral replication long-term.

One of the most promising approaches to halt new HIV infections is the use of antiretroviral therapy (ART) as pre-exposure prophylaxis (PrEP) to prevent HIV acquisition. ART is also used to treat HIV-positive individuals to suppress viral replication long-term. HIV-positive individuals on prolonged ART experience non-HIV related morbidities such as liver dysfunction, cardiovascular disease, bone disorders, and lipodystrophy. Similarly, use of ART in HIV-negative individuals as PrEP results in unwanted side effects that reduce adherence. Therefore, the development of new user-friendly PrEP and HIV suppression regimens, particularly with simplified dosing regimens that lack unwanted negative side effects, is an urgent global health need.

CCR5 is the main HIV entry co-receptor, and thus represents a promising antiviral therapeutic target. Sexual transmission of HIV is due to the transmission of only one or two clones, and these founder viruses almost universally utilize CCR5, not CXCR4, as the co-receptor. The reliance of transmitted founder HIV clones on the CCR5 co-receptor explains the extremely high level of resistance to HIV infection exhibited by individuals homozygous for the CCR5Δ32 allele. Furthermore, the only two known cases of HIV cure occurred in the setting of allogeneic stem cell transplantation using a stem cell graft from a CCR5^{Δ32/Δ32} homozygous donor. These characteristics suggest CCR5 is an ideal target for (1) pre-exposure prophylaxis (PrEP) to prevent HIV acquisition, and (2) for long-term functional cure through control of HIV replication and plasma viremia.

To this end, the present disclosure provides CCR5 binding agents that have been modified to increase effector function and circulation half-life for the prevention and treatment of HIV and methods of using the same.

### SUMMARY

The present disclosure provides CCR5 binding agents with increased effector function and circulation half-life that are useful for preventing and treating HIV. Also provided are methods for the prevention and treatment of HIV by administering to the patient, an effective amount of said CCR5 binding agents.

The present disclosure provides a method of preventing HIV infection in a subject comprising administering to the subject an effective amount of a CCR5 antibody comprising the following human IgG Fc amino acid substitutions: (i) M428L and N434S; (ii) L234A and L235A; and (iii) S131C wherein the antibody comprises: (a) a heavy chain variable region (VH) comprising a heavy chain complementary determining region 1 (HCDR1) of SEQ ID NO:11, a heavy chain complementary determining region 2 (HCDR2) of SEQ ID NO:12, and a heavy chain complementary determining region 3 (HCDR3) of SEQ ID NO:13; and (b) a light chain variable region (VL) comprising a light chain complementary determining region 1 (LCDR1) of SEQ ID NO:9, a light chain complementary determining region 2 (LCDR2) of SEQ ID NO:10, and a light chain complementary determining region 3 (LCDR3) of SEQ ID NO:11.

In some additional embodiments, the method may include one or more of the following additional elements, in any combinations unless clearly mutually exclusive: the CCR5 antibody is administered by injection;
the CCR5 antibody is administered subcutaneously;
the serum half-life of the CCR5 antibody is extended;
the serum concentration of the CCR5 antibody is increased;
the percentage of CCR5 receptors occupied by the CCR5 antibody on CCR5+ CD4+ T cells is increased;
the period of time in which CCR5 receptors are occupied by the CCR5 antibody on CCR5+ CD4+ T cells is extended;
the period of time in which CCR5 receptors are fully occupied by the CCR5 antibody on CCR5+ CD4+ T cells in the blood is at least 13 weeks;
the period of time in which CCR5 receptors are fully occupied by the CCR5 antibody on CCR5+ CD4+ T cells in the blood is at least 18 weeks;
the period of time in which CCR5 receptors are fully occupied by the CCR5 antibody on CCR5+ CD4+ T cells in the rectum is at least 15 weeks;
the period of time in which CCR5 receptors are fully occupied by the CCR5 antibody on CCR5+ CD4+ T cells in the vagina is at least 15 weeks;
the CCR5 antibody is administered at a dose of about 350 mg;
the CCR5 antibody is administered at a dose of about 525 mg;
the CCR5 antibody is administered at a dose of about 700 mg;
the CCR5 binding agent is administered at a dose of about 10 mg/kg; or the CCR5 binding agent is administered at approximately 12-week intervals.

The present disclosure also provides a method of conferring long-term CCR5 receptor occupancy (RO) in the vaginal or rectal tissue of a subject, the method comprising administering to the subject an effective amount of a CCR5 antibody comprising a human IgG Fc amino acid substitution, wherein the antibody comprises: (a) a heavy chain variable region (VH) comprising a heavy chain complementary determining region 1 (HCDR1) of SEQ ID NO:11, a heavy chain complementary determining region 2 (HCDR2) of SEQ ID NO:12, and a heavy chain complementary determining region 3 (HCDR3) of SEQ ID NO:13; and (b) a light chain variable region (VL) comprising a light chain complementary determining region 1 (LCDR1) of SEQ ID NO: 9, a light chain complementary determining region 2 (LCDR2) of SEQ ID NO:10, and a light chain complementary determining region 3 (LCDR3) of SEQ ID NO:11, wherein full receptor occupancy of CCR5 on CD4+ T cells in vaginal or rectal tissue of the subject persists for at least four weeks subsequent to administration.

In some additional embodiments, the vector may include one or more of the following additional elements, in any combinations unless clearly mutually exclusive:
full receptor occupancy of CCR5 on CD4+ T cells in vaginal or rectal tissue of the subject persists for at least twelve weeks subsequent to administration;
full receptor occupancy of CCR5 on CD4+ T cells in vaginal or rectal tissue of the subject persists for at least fifteen weeks subsequent to administration; or
the human IgG Fc amino acid substitutions comprise M428L and N434S, L234A and L235A, and S131C.

The present disclosure also provides a method of conferring long-term CCR5 receptor occupancy (RO) in the peripheral blood cells of a subject, the method comprising administering to the subject an effective amount of a CCR5 antibody comprising a human IgG Fc amino acid substitution, wherein the antibody comprises: (a) a heavy chain variable region (VH) comprising a heavy chain complementary determining region 1 (HCDR1) of SEQ ID NO: 11, a heavy chain complementary determining region 2 (HCDR2) of SEQ ID NO:12, and a heavy chain complementary determining region 3 (HCDR3) of SEQ ID NO:13; and (b) a light chain variable region (VL) comprising a light chain complementary determining region 1 (LCDR1) of SEQ ID NO:9, a light chain complementary determining region 2 (LCDR2) of SEQ ID NO: 10, and a light chain complementary determining region 3 (LCDR3) of SEQ ID NO:11, wherein full receptor occupancy of CCR5 on CD4+ peripheral blood T cells of the subject persists for at least thirteen weeks subsequent to administration.

In some additional embodiments, the vector may include one or more of the following additional elements, in any combinations unless clearly mutually exclusive:
full receptor occupancy of CCR5 on CD4+ T cells in vaginal or rectal tissue of the subject persists for at least twenty weeks subsequent to administration; or
the human IgG Fc amino acid substitutions comprise M428L and N434S, L234A and L235A, and S131C.

### BRIEF DESCRIPTION OF THE DRAWINGS

FIGS. 1A-1D show pharmacokinetics of leronlimab in rhesus macaques. Four rhesus macaques were administered a single 10 mg/kg subcutaneous dose of leronlimab (human IgG4 Fc). Leronlimab in plasma (FIG. 1A, reported in μg/ml), anti-drug antibody in plasma (FIG. 1B, reported as reciprocal dilution), and CCR5 receptor occupancy in blood (FIG. 1C) and rectal (FIG. 1D) CCR5+ CD4+ T cells is shown.

FIGS. 2A-2D show leronlimab levels in rectal and vaginal tissues in rhesus macaques. Four rhesus macaques were administered a single 10 mg/kg subcutaneous dose of leronlimab (human IgG4 Fc). Leronlimab was quantitated longitudinally in rectal tissue (FIG. 2A), vaginal tissue (FIG. 2B), rectal swabs (FIG. 2C), and vaginal swabs (FIG. 2D).

FIGS. 3A-3E show pharmacokinetics of macaque IgG4 LS leronlimab (“MacLS”) in rhesus macaques. Four rhesus macaques were administered a single 10 mg/kg subcutaneous dose of MacLS. Leronlimab in plasma (FIG. 3A, reported in μg/ml), anti-drug antibody in plasma (FIG. 3B, reported as reciprocal dilution), and CCR5 receptor occupancy in blood (FIG. 3C), rectal (FIG. 3D), and vaginal (FIG. 3E) CCR5+ CD4+ T cells is shown.

FIGS. 4A-4D show macaque IgG4 LS leronlimab (“MacLS”) levels in rectal and vaginal tissues in rhesus macaques. Four rhesus macaques were administered a single 10 mg/kg subcutaneous dose of MacLS. MacLS was quantitated longitudinally in rectal tissue (FIG. 4A), vaginal tissue (FIG. 4B), rectal swabs (FIG. 4C), and vaginal swabs (FIG. 4D).

FIGS. 5A-5E show pre-exposure prophylactic (PrEP) activity of macaque IgG4 LS leronlimab (“MacLS”) in an in vivo rhesus macaque model of simian HIV (SHIV) infection. Six rhesus macaques were administered a single 10 mg/kg subcutaneous dose of MacLS. Starting at one week post-MacLS, animals were challenged weekly at a low dose, intra-rectally with SHIV_{SF162P3}. Leronlimab in plasma (FIG. 5A, reported in μg/ml), anti-drug antibody in plasma (FIG. 5B, reported as reciprocal dilution), CCR5 receptor occupancy in blood CCR5+ CD4+ T cells (FIG. 5C), and SHIV viral titer in plasma (FIG. 5D, reported as viral RNA copies/mL) are shown. FIG. 5E shows viral acquisition in macaques treated with MacLS versus control macaques. Control animals included two macaques challenged concurrently and six historical control macaques that were challenged with the same virus stock at the same dose. P value was calculated by log-rank (Mantel-Cox) test.

### DETAILED DESCRIPTION

The present disclosure provides CCR5 binding agents, e.g., leronlimab, that are useful in preventing and treating HIV infection in patients wherein the CCR5 binding agent has been modified to increase effector function and circulation half-life and methods of use thereof.

Prior to setting forth this disclosure in more detail, it may be helpful to an understanding thereof to provide definitions of certain terms to be used herein. Unless defined otherwise, all technical and scientific terms used herein have the same meaning as is commonly understood by one of skill in the art to which this invention belongs. Additional definitions are set forth throughout this disclosure.

In the present description, the term “about” means ±20% of the indicated range, value, or structure, unless otherwise indicated. The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof. Any ranges provided herein include all the values and narrower ranges in the ranges.

As used herein, “chemokine” refers to a low-molecular weight cytokine that can stimulate recruitment of leukocytes. Chemokines have cysteine residues in conserved locations that are key to forming their 3-dimensional shape. Chemokines may be classified into four main subfamilies: Cys-Cys (C-C), Cys-X-Cys (CXC), CX₃C, and XC depending on the spacing of their first two amino terminal cysteine residues. Chemokines may also be grouped according to their function, such as whether they are inflammatory or homeostatic. There are 47 known chemokines, including but not limited to CCL5 (also known as RANTES), MIP-1α, MIP-1β, or SDF-1, or another chemokine which has similar activity.

As used herein, “C-C chemokine receptor 5,” also known as “CCR5” or “CD195” refers to a G protein-coupled receptor expressed on lymphocytes (e.g., NK cells, B cells, T cells), monocytes, dendritic cells, eosinophils, and microglia, which functions as a chemokine receptor for the C-C chemokine group. CCR5's cognate ligands include CCL3, CCL4, CCL3L1, and CCL5. In some embodiments, CCR5 refers to human CCR5. In some embodiments, CCR5 refers to a protein having an amino acid sequence provided in NCBI Reference Sequence: NP_000570.1 (SEQ ID NO:15).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, Y-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

As used herein, “protein” or “polypeptide” as used herein refers to a compound made up of amino acid residues that are covalently linked by peptide bonds. The term “protein” may be synonymous with the term “polypeptide” or may refer, in addition, to a complex of two or more polypeptides. A polypeptide may further contain other components (e.g., covalently bound), such as a tag, a label, a bioactive molecule, or any combination thereof. In certain embodiments, a polypeptide may be a fragment. As used herein, a “fragment” means a polypeptide that is lacking one or more amino acids that are found in a reference sequence. A fragment can comprise a binding domain, antigen, or epitope found in a reference sequence. A fragment of a reference polypeptide can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of amino acids of the amino acid sequence of the reference sequence.

As described herein, a “variant” polypeptide species has one or more non-natural amino acids, one or more amino acid substitutions, one or more amino acid insertions, one or more amino acid deletions, or any combination thereof at one or more sites relative to a reference polypeptide as presented herein. In certain embodiments, “variant” means a polypeptide having a substantially similar activity (e.g., enzymatic function, immunogenicity) or structure relative to a reference polypeptide). A variant of a reference polypeptide can have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to the amino acid sequence for the reference polypeptide as determined by sequence alignment programs and parameters known in the art. The variant can result from, for example, a genetic polymorphism or human manipulation. Conservative substitutions of amino acids are well known and may occur naturally or may be introduced when a protein is recombinantly produced. Amino acid substitutions, deletions, and additions may be introduced into a protein using mutagenesis methods known in the art (see, e.g., Sambrook et al., Molecular Cloning: A Laboratory Manual, 3d ed., Cold Spring Harbor Laboratory Press, NY, 2001). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare polypeptide variants (see, e.g., Sambrook et al., supra).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

The terms “identical” or “percent identity,” in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using methods known in the art, such as a sequence comparison algorithm, by manual alignment, or by visual inspection. The algorithm used herein for determining percent sequence identity and sequence similarity is the BLAST 2.0 algorithm, as described in Altschul et al. “Gapped BLAST and PSI-BLAST: a new generation of protein database search programs,” Nucleic Acids Res. 2007, 25, 3389-3402. Within the context of this disclosure, it will be understood that where sequence analysis software is used for analysis, the results of the analysis are based on the “default values” of the program referenced. “Default values” mean any set of values or parameters which originally load with the software when first initialized.

As used herein, a “fusion protein” comprises a single chain polypeptide having at least two distinct domains, wherein the domains are not naturally found together in a protein. A nucleic acid molecule encoding a fusion protein may be constructed using PCR, recombinantly engineered, or the like, or such fusion proteins can be made synthetically. A fusion protein may further contain other components (e.g., covalently bound), such as a tag, linker, transduction marker, or bioactive molecule.

A “nucleic acid molecule” or “polynucleotide” refers to a polymeric compound containing nucleotides that are covalently linked by 3′-5′ phosphodiester bonds. Nucleic acid molecules include polyribonucleic acid (RNA), polydeoxyribonucleic acid (DNA), which includes genomic DNA, mitochondrial DNA, cDNA, or vector DNA. A nucleic acid molecule may be double stranded or single stranded, and if single stranded, may be the coding strand or non-coding (anti-sense strand). A nucleic acid molecule may contain natural subunits or non-natural subunits. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of the polynucleotides of this disclosure are also contemplated. Variant polynucleotides are at least 80%, 85%, 90%, 95%, 99%, or 99.9% identical to a reference polynucleotide as described herein, or that hybridizes to a reference polynucleotide of defined sequence under stringent hybridization conditions of 0.015M sodium chloride, 0.0015M sodium citrate at about 65°−68° C. or 0.015M sodium chloride, 0.0015M sodium citrate, and 50% formamide at about 42° C. The polynucleotide variants retain the capacity to encode an immunoglobulin-like binding protein or antigen-binding fragment thereof having the functionality described herein.

The term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the co-existing materials in the natural system, is isolated. Such polynucleotide could be part of a vector and/or such polynucleotide or polypeptide could be part of a composition (e.g., a cell lysate), and still be isolated in that such vector or composition is not part of the natural environment for the nucleic acid or polypeptide.

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “exogenous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous or exogenous nucleic acid molecule, construct or sequence may be from a different genus or species. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein, or other activity encoded by an exogenous nucleic acid molecule introduced into the host cell, even if the host cell encodes a homologous protein or activity.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

As used herein, the term “expression”, refers to the process by which a polypeptide is produced based on the encoding sequence of a nucleic acid molecule, such as a gene. The process may include transcription, post-transcriptional control, post-transcriptional modification, translation, post-translational control, posttranslational modification, or any combination thereof. An expressed nucleic acid molecule is typically operably linked to an expression control sequence (e.g., a promoter).

As used herein, the term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

As used herein, the term “host” refers to a cell (e.g., T cell, Chinese Hamster Ovary (CHO) cell, HEK293 cell, B cell, or the like) or microorganism targeted for genetic modification with a heterologous nucleic acid molecule to produce a polypeptide of interest (e.g., a CCR5 antibody of the present disclosure). In certain embodiments, a host cell may optionally already possess or be modified to include other genetic modifications that confer desired properties related or unrelated to, e.g., biosynthesis of the heterologous protein (e.g., inclusion of a detectable marker; deleted, altered or truncated endogenous BCR).

As described herein, more than one heterologous nucleic acid molecule can be introduced into a host cell as separate nucleic acid molecules, as a plurality of individually controlled genes, as a polycistronic nucleic acid molecule (e.g., a heavy chain and a light chain of an antibody), as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “introduced” in the context of inserting a nucleic acid sequence into a cell, means “transfection”, or “transformation” or “transduction” and includes reference to the incorporation of a nucleic acid sequence into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

“Treat” or “treatment” or “ameliorate” refers to medical management of a disease, disorder, or condition of a patient (e.g., a human or non-human mammal, such as a primate, horse, cat, dog, goat, mouse, or rat). In general, an appropriate dose or treatment regimen comprising leronlimab is administered in an amount sufficient to elicit a therapeutic effect or therapeutic benefit. Treating an HIV infection in a subject refers to slowing, stopping or reversing the progression of an HIV disorder in the subject. In a preferred embodiment, “treating” refers to reversing the progression to the point of eliminating the disorder. As used herein, “treating” also means reducing the number of viral infections, reducing the number of infectious viral particles, reducing the number of virally infected cells, or ameliorating symptoms associated with HIV. Reducing viral load in a subject is one embodiment of treating the subject.

A prophylactic treatment meant to “prevent” a disease or condition (e.g., HIV infection in a patient) is a treatment administered to a patient who does not exhibit signs of a disease or exhibits only early signs, for the purpose of decreasing the risk of developing pathology or further advancement of the early disease. Such a prophylactic treatment may be referred to as “pre-exposure prophylaxis” (PrEP). For example, if an individual at risk of contracting HIV is treated with the methods of the present disclosure and does not contract HIV, then the disease has been prevented, at least over a period of time, in that individual.

A “therapeutically effective amount” or “effective amount” of leronlimab refers to an amount of leronlimab sufficient to result in a therapeutic effect, including improved clinical outcome; lessening or alleviation of symptoms associated with a disease; modulating immune response to lessen, reduce, or dampen counterproductive inflammatory cytokine activity; modulating immune response to normalize counterproductive inflammatory cytokine activity; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian patient and generally recognized as safe or not causing a serious adverse event.

The terms “CCR5 receptor occupancy” and “CCR5 RO” refer to the percentage of CCR5 RO on the surface of CD4+ T cells. An equation is used to measure unoccupied CCR5 receptors by using Pacific Blue-conjugated Leronlimab (termed Leronlimab-PB). CCR5 RO is defined as the percentage of cells CCR5+ (measured by non-competing alternative clone 3A9 or J418) and Leronlimab+ (measured by anti-human IgG4 or anti-rhesus IgG4, depending in the leronlimab version used) divided by the percentage of cells CCR5+ and Leronlimab+ (measured by the sum of anti-human or rhesus IgG4 and Leronlimab-PB) cells following incubation with a saturating concentration of Leronlimab-PB.

Additional definitions are provided in the sections below.

### CCR5 Binding Agents

The present disclosure provides for use of a CCR5 binding agent, e.g., leronlimab, or antigen binding fragment thereof, in the prevention and treatment of HIV. CCR5 binding agents for use in the present disclosure are inhibitors of CCR5 activity induced by CCL5 binding. The term “inhibit” or “inhibitor” refers to a diminishing, blunting, reduction, masking, interrupting, blocking, mitigation, or slowing directly or indirectly, in the expression, amount or activity of a target or signaling pathway relative to (1) a control, endogenous or reference target or pathway, or (2) the absence of a target or pathway, wherein the diminishing, blunting, reduction, masking, interrupting, blocking, mitigation, or slowing is statistically, biologically, or clinically significant. For example, an inhibitor of CCR5 may diminish, blunt, reduce, mask, interrupt, block, mitigate, or slow CCR5 signaling activity induced by CCL5 binding by about 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated CCR5. CCR5 activity induced by CCL5 binding may be measured by detecting, for example, a decrease in cAMP, cell migration, or both.

The CCR5 receptor is a C-C chemokine G-coupled protein receptor expressed on lymphocytes (e.g., NK cells, B cells), monocytes, monocytes, dendritic cells, a subset of T cells, etc. The extracellular portions represent potential targets for antibodies targeting CCR5, and comprise an amino-terminal domain (Nt) and three extracellular loops (ECL1, ECL2, and ECL3). The extracellular portions of CCR5 comprise just 90 amino acids distributed over four domains. The largest of these domains are at the Nt and ECL2 at approximately 30 amino acids each (Olson et al., Curr. Opin. HIV AIDS, March 4 (2): 104-111 (2009)).

The CCR5 receptor binds to a chemokine known as CCL5 (C-C chemokine ligand 5), which is an inflammatory chemokine that plays an important role in immunologic mechanisms such as controlling cell recruitment and activation in basal and inflammatory circumstances. CCL5 acts as a key regulator of CCR5+ cell (e.g., monocyte and T cell) migration to inflammatory sites, directing migration of monocytes and T cells to damaged or infected sites. CCR5 also plays a crucial role in differentiation and activation of CD8+ T cells. Many biologic effects of chemokines are mediated by their interaction with chemokine receptors on cell surfaces. The most relevant known receptor for CCL5 is the CCR5 receptor; however, CCR1 and CCR3 are also known CCL5 receptors and CCR4 and CD44 are auxiliary receptors. Tamamis et al., Elucidating a Key Anti-HIV-1 and Cancer-Associated Axis: The Structure of CCL5 (Rantes) in Complex with CCR5, SCIENTIFIC REPORTS, 4:5447 (2014).

The formation of the CCL5 ligand and CCR5 receptor complex causes a conformational change in the receptor that activates the subunits of the G-protein, inducing signaling and leading to changed levels of cyclic AMP (CAMP), inositol triphosphate, intracellular calcium and tyrosine kinase activation. These signaling events cause cell polarization and translocation of the transcription factor NF-kB, which results in the increase of phagocytic ability, cell survival, and transcription of proinflammatory genes.

CCR5 binding agents include, but are not limited to, small molecules, antibodies or antigen binding fragments thereof, proteins, peptides, nucleic acids, and aptamers.

In some embodiments, a CCR5 binding agent is an anti-CCR5 antibody or antigen-binding fragment thereof that specifically binds to CCR5, e.g., to an epitope on CCR5. The term “epitope” or “antigenic epitope” includes any molecule, structure, amino acid sequence, or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

As used herein, “specifically binds” or “specific for” may in some embodiments refer to an association or union of a binding protein (e.g., an anti-CCR5 antibody) or a binding domain (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding domains (or fusion proteins thereof) may be classified as “high affinity” binding domains (or fusion proteins thereof) and “low affinity” binding domains (or fusion proteins thereof). “High affinity” binding domains refer to those binding domains with a Kₐ of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Kₐ of up to 10⁸ M⁻¹, up to 10⁷ M⁻¹, up to 10⁶ M⁻¹, up to 10⁵ M⁻¹. Alternatively, affinity may be defined as an equilibrium dissociation constant (K_{D}) of a particular binding interaction with units of M (e.g., 10⁻⁵ M to 10⁻¹³ M), (which equals the ratio of the off-rate [k_{off}] to the on-rate [kₒₙ] for this association reaction).

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

Terms understood by those in the art of antibody technology are each given the meaning acquired in the art, unless expressly defined differently herein. The term “antibody” refers to an intact antibody comprising at least two heavy (H) chains and two light (L) chains inter-connected by disulfide bonds, as well as any antigen-binding portion or fragment of an intact antibody, such as an scFv, Fab, or Fab′2 fragment, that has or retains the ability to bind to the antigen target molecule recognized by the intact antibody. Thus, the term “antibody” herein is used in the broadest sense and includes polyclonal and monoclonal antibodies, including intact antibodies and functional (antigen-binding) antibody fragments thereof, including fragment antigen binding (Fab) fragments, F (ab′) 2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody). The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

An anti-CCR5 monoclonal antibody or antigen-binding portion thereof for use in the methods disclosed herein may be non-human (e.g., murine), chimeric, humanized, or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

The terms “VL” and “VH” refer to the variable binding region from an antibody light chain and an antibody heavy chain, respectively. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary amino acid sequence by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

Numbering of CDR and framework regions may be determined according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Plückthun, J. Mol. Bio. 309:657-670 (2001)). Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme.

In some embodiments, the present disclosure provides use of an anti-CCR5 antibody or antigen binding fragment thereof having a light chain variable region (VL) that is at least 70% identical to SEQ ID NO: 1, at least 75% identical to SEQ ID NO: 1, at least 80% identical to SEQ ID NO: 1, at least 85% identical to SEQ ID NO: 1, or at least 90% identical to SEQ ID NO: 1. In some embodiments, the present disclosure provides use of an anti-CCR5 antibody or antigen binding fragment thereof having a light chain variable antibody region that is 70%-100% identical to SEQ ID NO: 1, 75%-100% identical to SEQ ID NO: 1, 80%-100% identical to SEQ ID NO: 1, 85%-100% identical to SEQ ID NO: 1, 90%-100% identical to SEQ ID NO: 1 or 91%-100% identical to SEQ ID NO: 1.

In some embodiments, the present disclosure provides use of an anti-CCR5 antibody or antigen binding fragment thereof having a light chain variable region (VL) that is at least 70% identical to amino acids 20-131 of SEQ ID NO: 1, at least 75% identical to amino acids 20-131 of SEQ ID NO: 1, at least 80% identical to amino acids 20-131 of SEQ ID NO: 1, at least 85% identical to amino acids 20-131 of SEQ ID NO: 1, or at least 90% identical to amino acids 20-131 of SEQ ID NO: 1. In some embodiments, the present disclosure provides use of an anti-CCR5 antibody or antigen binding fragment thereof having a light chain variable antibody region that is 70%-100% identical to amino acids 20-131 of SEQ ID NO: 1, 75%-100% identical to amino acids 20-131 of SEQ ID NO: 1, 80%-100% identical to amino acids 20-131 of SEQ ID NO: 1, 85%-100% identical to amino acids 20-131 of SEQ ID NO: 1, 90%-100% identical to amino acids 20-131 of SEQ ID NO: 1 or 91%-100% identical to amino acids 20-131 of SEQ ID NO: 1.

In some embodiments, the present disclosure provides use of an anti-CCR5 antibody or antigen binding fragment thereof having a heavy chain variable region (VH) that is at least 70% identical to SEQ ID NO:3, at least 75% identical to SEQ ID NO:3, at least 80% identical to SEQ ID NO:3, at least 85% identical to SEQ ID NO:3, or at least 90% identical to SEQ ID NO: 3. In some embodiments the present disclosure provides use of an anti-CCR5 antibody or antigen binding fragment thereof having a heavy chain antibody variable region that is 70%-100% identical to SEQ ID NO: 3, 75%-100% identical to SEQ ID NO: 3, 80%-100% identical to SEQ ID NO: 3, 85%-100% identical to SEQ ID NO: 3, 90%-100% identical to SEQ ID NO: 3, or 91%-100% identical to SEQ ID NO:3.

In some embodiments, the present disclosure provides use of an anti-CCR5 antibody or antigen binding fragment thereof having a heavy chain variable region (VH) that is at least 70% identical to amino acids 20-141 of SEQ ID NO:3, at least 75% identical to amino acids 20-141 of SEQ ID NO:3, at least 80% identical to amino acids 20-141 of SEQ ID NO:3, at least 85% identical to amino acids 20-141 of SEQ ID NO:3, or at least 90% identical to amino acids 20-141 of SEQ ID NO:3. In some embodiments the present disclosure provides use of an anti-CCR5 antibody or antigen binding fragment thereof having a heavy chain antibody variable region that is 70%-100% identical to amino acids 20-141 of SEQ ID NO: 3, 75%-100% identical to amino acids 20-141 of SEQ ID NO: 3, 80%-100% identical to amino acids 20-141 of SEQ ID NO: 3, 85%-100% identical to amino acids 20-141 of SEQ ID NO: 3, 90%-100% identical to amino acids 20-141 of SEQ ID NO: 3, or 91%-100% identical to amino acids 20-141 of SEQ ID NO: 3.

In some embodiments, the present disclosure provides use of an anti-CCR5 antibody having a heavy chain variable region (VH) that is at least 70% identical to SEQ ID NO:5, at least 75% identical to SEQ ID NO: 5, at least 80% identical to SEQ ID NO: 5, at least 85% identical to SEQ ID NO: 5, or at least 90% identical to SEQ ID NO: 5. In some embodiments the present disclosure provides use of an anti-CCR5 antibody having a heavy chain variable antibody region that is 70%-100% identical to SEQ ID NO: 5, 75%-100% identical to SEQ ID NO: 5, 80%-100% identical to SEQ ID NO: 5, 85%-100% identical to SEQ ID NO: 5, 90%-100% identical to SEQ ID NO: 5, or 91%-100% identical to SEQ ID NO: 5.

In some embodiments, the present disclosure provides use of an anti-CCR5 antibody having a heavy chain variable region (VH) that is at least 70% identical to amino acids 20-141 of SEQ ID NO:5, at least 75% identical to amino acids 20-141 of SEQ ID NO: 5, at least 80% identical to amino acids 20-141 of SEQ ID NO: 5, at least 85% identical to amino acids 20-141 of SEQ ID NO: 5, or at least 90% identical to amino acids 20-141 of SEQ ID NO: 5. In some embodiments the present disclosure provides use of an anti-CCR5 antibody having a heavy chain variable antibody region that is 70%-100% identical to amino acids 20-141 of SEQ ID NO: 5, 75%-100% identical to amino acids 20-141 of SEQ ID NO: 5, 80%-100% identical to amino acids 20-141 of SEQ ID NO: 5, 85%-100% identical to amino acids 20-141 of SEQ ID NO: 5, 90%-100% identical to amino acids 20-141 of SEQ ID NO: 5, or 91%-100% identical to amino acids 20-141 of SEQ ID NO: 5.

In some embodiments, the present disclosure provides use of an anti-CCR5 antibody or an antigen-binding fragment thereof comprising a heavy chain variable region (VH) and a light chain variable region (VL), wherein the VH comprises a heavy chain CDR1 (VH-CDR1) comprising the amino acid sequence of SEQ ID NO: 12, a heavy chain CDR2 (VH-CDR2) comprising the amino acid sequence of SEQ ID NO:13, and a heavy chain CDR3 (VH-CDR3) comprising the amino acid sequence of SEQ ID NO:14; and the VL comprises a light chain CDR1 (VL-CDR1) comprising the amino acid sequence of SEQ ID NO:9, a light chain CDR2 (VL-CDR2) comprising the amino acid sequence of SEQ ID NO:10, and a light chain CDR3 (VL-CDR3) comprising the amino acid sequence of SEQ ID NO:11. In some such embodiments, the VH comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:3 or amino acids 20-141 of SEQ ID NO:3, and a VL comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO: 1 or amino acids 20-131 of SEQ ID NO: 1, provided that the amino acid sequences of the VH-CDRs (SEQ ID NOS: 12-14) and VL-CDRs (SEQ ID NOS: 9-11) are unchanged; or the VH comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:5 or amino acids 20-141 of SEQ ID NO:5, and a VL comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO: 1 or amino acids 20-131 of SEQ ID NO:1, provided that the amino acid sequences of the VH-CDRs (SEQ ID NOS: 12-14) and VL-CDRs (SEQ ID NOS: 9-11) are unchanged.

In some embodiments, the present disclosure provides use of an anti-CCR5 antibody or an antigen-binding fragment thereof comprising: (a) a VH comprising an amino acid sequence of SEQ ID NO:3 or amino acids 20-141 of SEQ ID NO:3, and a VL comprising an amino acid sequence of SEQ ID NO:1 or amino acids 20-131 of SEQ ID NO:1; or (b) a VH comprising an amino acid sequence of SEQ ID NO:5 or amino acids 20-141 of SEQ ID NO:5, and a VL comprising an amino acid sequence of SEQ ID NO:1 or amino acids 20-131 of SEQ ID NO:1.

In some embodiments, the present disclosure provides use of an anti-CCR5 antibody comprising a heavy chain (HC) and a light chain (LC). The heavy chain typically comprises a VH and a heavy chain constant region (CH). Depending on the antibody isotype from which it derives, a heavy chain constant region may comprise CH1, CH2, and CH3 domains (IgA, IgD, IgG), or CH1, CH2, CH3, and CH4 domains (IgE, IgM). In some embodiments, the heavy chain constant region comprises a human IgG1, IgG2, IgG3, or IgG4 constant region. In some embodiments, the constant region of the anti-CCR5 antibody is an IgG4 constant region. The light chain typically comprises a VL and a light chain constant region (CL). In some embodiments, a CL comprises a C kappa (“CK”) constant region. In some embodiments, a CL comprises a C lambda (Cλ) constant region. In some embodiments, an anti-CCR5 antibody of the present disclosure comprises two heavy chains and two light chains, held together covalently by disulfide bridges.

In some embodiments, the present disclosure provides use of an anti-CCR5 antibody comprising a heavy chain constant region portion having an effect on antibody stability by way of, for example, one or more amino acid substitutions or deletions in the CH1 region known in the art. An amino acid modification (e.g., substitution) to modify (e.g., improve, reduce, or ablate) antibody stability includes, for example, S131C.

The S131C mutation promotes the formation of disulfide bonds linking antibody heavy and light chains, which, in turn, helps prevent dissociation of the heavy and light chain after administration. This dissociation phenomena is sometimes referred to a Fab exchange and occurs in IgG4 molecules. Details of Fab exchange and the S131C mutation have been discussed in Aran F. Labrijn, et. al. “Species-Specific Determinants in the IgG CH3 Domain Enable Fab-Arm Exchange by Affecting the Noncovalent CH3-CH3 Interaction Strength” J. Immunol. 15 Sep. 2011; 187 (6): 3238-3246.

In some embodiments, the present disclosure provides use of an anti-CCR5 antibody comprising a Fc region portion. As used herein, “Fc region portion” refers to the heavy chain constant region segment of the Fc fragment (the “fragment crystallizable” region or Fc region) from an antibody, which can include one or more constant domains, such as CH2, CH3, CH4 or any combination thereof. In some embodiments, an Fc region portion includes the CH2 and CH3 domains of an IgG, IgA, or IgD antibody or any combination thereof, or the CH3 and CH4 domains of an IgM or IgE antibody, and any combination thereof. In some embodiments, a CH2CH3 or a CH3CH4 structure has sub-region domains from the same antibody isotype and are human, such as human IgG1, IgG2, IgG3, IgG4, IgA1, IgA2, IgD, IgE, or IgM (e.g., CH2CH3 from human IgG1). By way of background, an Fc region is responsible for the effector functions of an antibody, such as ADCC (antibody-dependent cell-mediated cytotoxicity), CDC (complement-dependent cytotoxicity) and complement fixation, binding to Fc receptors (e.g., CD16, CD32, FcRn), greater half-life in vivo relative to a polypeptide lacking an Fc region, protein A binding, and perhaps even placental transfer (see Capon et al. Nature 337:525, 1989). In some embodiments, a Fc region portion in an antibody or antigen-binding fragment of the present disclosure is capable of mediating one or more of these effector functions. In some embodiments, a Fc region portion in an antibody or antigen-binding fragment of the present disclosure has normal effector function, meaning having less than 20%, 15%, 10%, 5%, 1% difference in effector function (e.g., ADCC, CDC, half-life or any combination thereof) as compared to a wild type IgG1 antibody.

Amino acid modifications (e.g., substitutions) to modify (e.g., improve, reduce, or ablate) Fc functionalities include, for example, the M428L/N434S (“LS”) and L234A/L235A (“LALA”), mutations, which mutations are summarized and annotated in “Engineered Fc Regions”, published by InvivoGen (2011) and available online at InvitroGen's Engineer Fc Region web site, and are incorporated herein by reference.

In a preferred embodiment, the present disclosure provides use of an anti-CCR5 antibody comprising M428L/N434S, L234A/L235A, or S131C mutations, and any combinations thereof. The mutations may be made in a human IgG Fc, particularly a human IgG1 or human IgG4 Fc.

In some embodiments, the present disclosure provides use of an anti-CCR5 antibody that comprises a heavy chain (HC) and a light chain (LC), wherein the HC comprises an amino acid sequence that has at least 90%, 91%, 92%, 93%, 94%, 95%, 96%, 97%, 98%, 99%, or 100% identity with the amino acid sequence of SEQ ID NO:7, and the LC comprises an amino acid sequence that has at least 90%, 91%, 92%, 93%, 94%, 95%, 96%, 97%, 98%, or 99% identity with the amino acid sequence of SEQ ID NO:8

In some embodiments, the present disclosure provides use of an anti-CCR5 antibody that comprises a HC comprising an amino acid sequence that has the amino acid sequence of SEQ ID NO:7, and a LC comprising an amino acid sequence that has the amino acid sequence of SEQ ID NO:8.

In some embodiments, the present disclosure provides use of an anti-CCR5 antibody or antigen binding fragment thereof that are derivatized or otherwise modified. For example, derivatized antibodies can be modified by glycosylation, acetylation, pegylation, phosphorylation, amidation, derivatization by known protecting/blocking groups, proteolytic cleavage, or the like.

In any of the aforementioned embodiments, the anti-CCR5 antibody or antigen binding fragment thereof is conjugated to a small molecule drug to form an antibody drug conjugate.

In some embodiments, the present disclosure provides use of the monoclonal antibody PA14, produced by the hybridoma cell line designated PA14 (ATCC Accession No. HB-12610), or an antigen binding fragment thereof, or an antibody that competes with monoclonal antibody PA-14 in binding to CCR5.

In some embodiments, the present disclosure provides use of leronlimab (PRO140) antibody or antigen binding fragment thereof. Leronlimab (PRO140) is a humanized IgG4 monoclonal antibody that binds to CCR5 described in U.S. Pat. Nos. 7,122,185 and 8,821,877, which are incorporated herein by reference, in their entirety. Leronlimab (PRO 140) is a humanized version of the murine monoclonal antibody, PA14, which was generated against CD4⁺ CCR5⁺ cells. Olson et al., Differential Inhibition of Human Immunodeficiency Virus Type 1 Fusion, gp 120 Binding and CC-Chemokine Activity of Monoclonal Antibodies to CCR5, J. VIROL., 73:4145-4155. (1999). PRO 140 binds to CCR5 expressed on the surface of a cell, and potently inhibits HIV-1 entry and replication at concentrations that do not affect CCR5 chemokine receptor activity in vitro and in the hu-PBL-SCID mouse model of HIV-1 infection. Olson et al., Differential Inhibition of Human Immunodeficiency Virus Type 1 Fusion, gp 120 Binding and CC-Chemokine Activity of Monoclonal Antibodies to CCR5, J. VIROL., 73:4145-4155. (1999); Trkola et al., Potent, Broad-Spectrum Inhibition of Human Immunodeficiency Virus Type 1 by the CCR5 Monoclonal Antibody PRO 140, J. VIROL., 75:579-588 (2001). As used herein, unless context clearly indicates otherwise, “leronlimab” refers to antibodies having CDRs matching those of leronlimab as described in U.S. Pat. Nos. 7,122,185 and 8,821,877 and which may include any a Fc as conventionally known or of the various Fc modifications described herein.

Leronlimab does not downregulate CCR5 surface expression or deplete CCR5-expressing cells, but does prevent CCL5-induced calcium mobilization in CCR5+ cells with an IC₅₀ of 45 μg/ml. In some embodiments, a CCR5 binding agent does not downregulate CCR5 surface expression, deplete CCR5-expressing cells, or both. In some embodiments, a CCR5 binding agent inhibits CCL5-induced calcium mobilization of CCR5+ cells with an IC₅₀ of 45 μg/ml. In some embodiments, the CCR5 binding agent is leronlimab.

Leronlimab (PRO 140) binds to CCR5 and blocks viral entry by interfering with the final phase of viral binding to the cell surface prior to fusion of the viral and cell membranes. Leronlimab (PRO 140) has been administered intravenously or subcutaneously to more than 750 healthy and HIV-1 infected individuals in Phase I/II/III studies. The drug has been well tolerated following intravenous administration of single doses of 0.5 to 10 mg/kg or up to 700 mg weekly doses as subcutaneous (SC) injection. Overall, 324 patients have been exposed to leronlimab (PRO 140) 350 mg SC weekly dose with the longest duration of exposure lasting 4 years. Similarly, more than 250 and 150 patients have been exposed to leronlimab (PRO 140) 525 mg and 700 mg SC weekly dose, respectively.

In some embodiments, the present disclosure provides use of an anti-CCR5 antibody that binds to the same epitope as that to which leronlimab binds or competes with leronlimab in binding to CCR5. Leronlimab binds to a discontinuous epitope spanning multiple extracellular domains on CCR5, which include the N-terminus and second extracellular loop (ECL2) of CCR5 (Trkola et al. J. Virol. 75:579-588, incorporated by reference in its entirety). Leronlimab directly blocks binding of HIV Env to the CCR5 co-receptor via a competitive mechanism. Leronlimab binding at least requires amino acid residues D2 in the N-terminus and R168 and Y176 in the ECL2; mutation of amino acids D95 and C101 in the ECL1, and C178 in ECL2 also affect leronlimab binding, e.g., by conformational perturbation (Olson et al. J. Virol. 73:4145-4155, incorporated by reference in its entirety). Targeted loss-of-function mutagenesis and subsequent photo-cross-linking using genetically encoded unnatural amino acids method was also used to map antibody-GPCR complexes and identified residues 174 and 175 at the amino-terminal end of ECL2 as forming the strongest links with leronlimab (Ray-Saha et al., Biochem. 53:1302-13010).

CCR5 amino acid residues that are involved in CCL5 (RANTES) binding include K1, D2, D11, E18, K26 in the N-terminus, D95 in the ECL1, and K171, K191, and R274 in the ECL2 (Navenot et al. J. Mol. Biol. 313:1181-1193, incorporated by reference in its entirety).

Nucleic acids encoding heavy and light chains of the humanized PA14 antibodies have been deposited with the ATCC. Specifically, the plasmids designated pVK-HuPRO140, pVg4-HuPRO140 (mut B+D+I) and pVg4-HuPRO140 HG2, respectively, were deposited pursuant to, and in satisfaction of, the requirements of the Budapest Treaty with the ATCC, Manassas, Va., U.S.A. 20108, on Feb. 22, 2002, under ATCC Accession Nos. PTA 4097, PTA 4099, and PTA 4098, respectively. The American Type Culture Collection (ATCC) is now located at 10801 University Boulevard, Manassas, Va. 20110-2209. The plasmids designated pVK-HuPRO140 and pVg4-HuPRO140 HG2 encode the light chain and heavy chain, respectively, of leronlimab.

The HCDR1-3 and LCDR1-3 amino acid sequences of leronlimab are set forth in SEQ ID NOS: 12-14 and 9-11, respectively. The heavy chain and light chain sequences of leronlimab are set forth in SEQ ID NOS: 7 and 8, respectively.

Leronlimab sequences with leader peptides, which are typically cleaved during final antibody processing are found in SEQ ID NOs: 19 and 20, with encoding nucleotide sequences in SEQ ID NOs: 21 and 22.

In some embodiments, the present disclosure provides use of a CCR5 binding agent that is a competitive inhibitor to CCR5. The term “competitive inhibitor” as used herein refers to a molecule that competes with a reference molecule for binding to a target, and thereby blunts, inhibits, dampens, reduces, or blocks the effects of the reference molecule on the target. Thus a competitive inhibitor to CCR5 would compete with CCL5 for binding to CCR5. In some embodiments, a competitive inhibitor to CCR5 is an antibody or antigen binding fragment thereof that comprises:
(i) a heavy chain variable region (VH) and a light chain variable region (VL), wherein the VH comprises a heavy chain CDR1 (VH-CDR1) comprising the amino acid sequence of SEQ ID NO:12, a heavy chain CDR2 (VH-CDR2) comprising the amino acid sequence of SEQ ID NO: 13, and a heavy chain CDR3 (VH-CDR3) comprising the amino acid sequence of SEQ ID NO: 14; and the VL comprises a light chain CDR1 (VL-CDR1) comprising the amino acid sequence of SEQ ID NO:9, a light chain CDR2 (VL-CDR2) comprising the amino acid sequence of SEQ ID NO:10, and a light chain CDR3 (VL-CDR3) comprising the amino acid sequence of SEQ ID NO:11;
(ii) a VH comprising the amino acid sequence of SEQ ID NO:3 or amino acids 20-141 of SEQ ID NO:3, and a VL comprising the amino acid sequence of SEQ ID NO:1 or amino acids 20-131 of SEQ ID NO:1;
(iii) a VH comprises the amino acid sequence of SEQ ID NO:5 or amino acids 20-141 of SEQ ID NO:5, and a VL comprises the amino acid sequence of SEQ ID NO: 1 or amino acids 20-131 of SEQ ID NO:1; or
(iv) a heavy chain comprising the amino acid sequence of SEQ ID NO:7 and a light chain comprising the amino acid sequence of SEQ ID NO:8.

In some embodiments, the present disclosure provides use of an anti-CCR5 antibody or an antigen-binding fragment thereof comprising: (a) a VH comprising an amino acid sequence of SEQ ID NO:3 or amino acids 20-141 of SEQ ID NO:3, and a VL comprising an amino acid sequence of SEQ ID NO: 1 or amino acids 20-131 of SEQ ID NO:1; or (b) a VH comprising an amino acid sequence of SEQ ID NO:5 or amino acids 20-141 of SEQ ID NO:5, and a VL comprising an amino acid sequence of SEQ ID NO: 1 or amino acids 20-131 of SEQ ID NO:1.

In some embodiments, the present disclosure provides use of a CCR5 binding agent that is a competitive inhibitor to CCR5 and does not have CCL5 agonist activity upon binding to CCR5. CCL5 agonist activity may be detected by measuring a decrease in cAMP; induced cell migration; or both cAMP decrease and induced cell migration triggered in response to CCL5-CCR5 axis activity. Some CCR5 binding agents even while acting to inhibit, interrupt, block, mitigate, dampen, slow the progress of, or eliminate the triggering of the downstream effects of CCL5 on CCR5 receptor positive cells also gives rise to independent and separate CCL5 agonistic downstream CCL5/CCR5 axis signaling effects that may counteract or diminish the effectiveness of these CCR5 competitive inhibitors for the purposes of immunomodulatory regulation, alteration, or control for therapeutic purposes. In some embodiments, a competitive inhibitor to CCR5 that does not have CCL5 agonist activity is an antibody or antigen binding fragment thereof that comprises:
(i) a heavy chain variable region (VH) and a light chain variable region (VL), wherein the VH comprises a heavy chain CDR1 (VH-CDR1) comprising the amino acid sequence of SEQ ID NO:12, a heavy chain CDR2 (VH-CDR2) comprising the amino acid sequence of SEQ ID NO: 13, and a heavy chain CDR3 (VH-CDR3) comprising the amino acid sequence of SEQ ID NO: 14; and the VL comprises a light chain CDR1 (VL-CDR1) comprising the amino acid sequence of SEQ ID NO:9, a light chain CDR2 (VL-CDR2) comprising the amino acid sequence of SEQ ID NO: 10, and a light chain CDR3 (VL-CDR3) comprising the amino acid sequence of SEQ ID NO:11;
(ii) a VH comprising the amino acid sequence of SEQ ID NO:3 or amino acids 20-141 of SEQ ID NO:3, and a VL comprising the amino acid sequence of SEQ ID NO:1 or amino acids 20-131 of SEQ ID NO:1;
(iii) a VH comprises the amino acid sequence of SEQ ID NO:5 or amino acids 20-141 of SEQ ID NO:5, and a VL comprises the amino acid sequence of SEQ ID NO: 1 or amino acids 20-131 of SEQ ID NO:1; or
(iv) a heavy chain comprising the amino acid sequence of SEQ ID NO:7 and a light chain comprising the amino acid sequence of SEQ ID NO:8.

### Nucleic Acids, Vectors, and Host Cells

In another aspect, the present disclosure provides an isolated nucleic acid that encodes the anti-CCR5 antibody or antigen binding fragment thereof as described herein. In some embodiments, the isolated nucleic acid encodes the VH, the VL, or both the VH and VL of the antibody or antigen binding fragment thereof. In some embodiments, the isolated nucleic acid encodes the heavy chain, the light chain, or both the heavy and light chain of the antibody or antigen binding fragment thereof. In some embodiments, the nucleic acid encoding the anti-CCR5 antibody or antigen binding fragment thereof is codon optimized to enhance or maximize expression in certain types of cells (e.g., Scholten et al., Clin. Immunol. 119:135-145, 2006). As used herein a “codon optimized” polynucleotide is a heterologous polypeptide having codons modified with silent mutations corresponding to the abundances of host cell tRNA levels.

In some embodiments, a nucleic acid molecule encoding an anti-CCR5 antibody or antigen binding fragment thereof of the present disclosure (e.g., an antibody heavy chain and light chain, or VH and VL regions) comprises a nucleic acid sequence for a heavy chain or VH region and a light chain or VL, respectively, wherein the heavy chain or VH region is separated from the light chain or VL region by a 2A self-cleaving peptide. In some embodiments, the 2A self-cleaving peptide is a porcine teschovirus-1 (P2A), equine rhinitis A virus (E2A), Thosea asigna virus (T2A), foot-and-mouth disease virus (F2A), or any combination thereof (see, e.g., Kim et al., PLOS One 6: e18556, 2011, which 2A nucleic acid and amino acid sequences are incorporated herein by reference in their entirety).

In another aspect, an expression construct comprising a nucleic acid encoding an anti-CCR5 antibody or antigen binding fragment thereof as described herein is provided. In some embodiments, a nucleic acid may be operably linked to an expression control sequence (e.g., expression construct). As used herein, “expression construct” refers to a DNA construct containing a nucleic acid molecule that is operably-linked to a suitable control sequence capable of effecting the expression of the nucleic acid molecule in a suitable host. An expression construct may be present in a vector (e.g., a bacterial vector, a viral vector) or may be integrated into a genome. The term “operably linked” refers to the association of two or more nucleic acids on a single polynucleotide fragment so that the function of one is affected by the other. For example, a promoter is operably-linked with a coding sequence when it is capable of affecting the expression of that coding sequence (i.e., the coding sequence is under the transcriptional control of the promoter). The term “expression control sequence” (also called a regulatory sequence) refers to nucleic acid sequences that effect the expression and processing of coding sequences to which they are operably linked. For example, expression control sequences may include transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion.

In some embodiments, a nucleic acid or an expression construct encoding an anti-CCR5 antibody or antigen binding fragment thereof is present in a vector. A “vector” is a nucleic acid molecule that is capable of transporting another nucleic acid. Vectors may be, for example, plasmids, cosmids, viruses, a RNA vector or a linear or circular DNA or RNA molecule that may include chromosomal, non-chromosomal, semi-synthetic or synthetic nucleic acids. Exemplary vectors are those capable of autonomous replication (episomal vector) or expression of nucleic acids to which they are linked (expression vectors). Exemplary viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). In some embodiments, a vector is a plasmid. In some other embodiments, a vector is a viral vector. In some such embodiments, the viral vector is a lentiviral vector or a γ-retroviral vector.

In a further aspect, the present disclosure also provides an isolated host cell comprising a nucleic acid, expression construct, or vector encoding an anti-CCR5 antibody or antigen binding fragment thereof as described herein. As used herein, the term “host” refers to a cell or microorganism targeted for genetic modification with a heterologous or exogenous nucleic acid molecule to produce a polypeptide of interest (e.g., an anti-CCR5 antibody or antigen-binding fragment thereof). In certain embodiments, a host cell may optionally already possess or be modified to include other genetic modifications that confer desired properties related or unrelated to biosynthesis of the heterologous or exogenous protein (e.g., inclusion of a selectable marker). More than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more exogenous nucleic acid molecules are introduced into a host cell, it is understood that the two more exogenous nucleic acid molecules can be introduced as a single nucleic acid molecule (e.g., on a single vector), on separate vectors, integrated into the host chromosome at a single site or multiple sites. The number of referenced heterologous nucleic acid molecules or protein activities refers to the number of encoding nucleic acid molecules or the number of protein activities, not the number of separate nucleic acid molecules introduced into a host cell.

Examples of host cells include, but are not limited to, eukaryotic cells, e.g., yeast cells, animal cells, insect cells, plant cells; and prokaryotic cells, including E. coli. In some embodiments, the cells are mammalian cells. In some embodiments, the host cell is a human embryonic kidney (HEK293) cell, Y0 cell, Sp2/0 cell, NS0 murine myeloma cell, PER.C6® human cell, baby hamster kidney cell (BHK), COS cell, or Chinese hamster ovary (CHO) cell. In some embodiments, the host cell is a CHO-K1 cell. In some embodiments, the host cell is a CHOK1SV cell. Host cells are cultured using methods known in the art.

In yet another aspect, the present disclosure provides a process for making an anti-CCR5 antibody or antigen binding fragment thereof as described herein, comprising culturing a host cell of the present disclosure, under suitable conditions and for a sufficient time to express the anti-CCR5 antibody or antigen binding fragment thereof, and optionally isolating the anti-CCR5 antibody or antigen binding fragment thereof from the culture. Purification of soluble antibodies or antigen binding fragments thereof may be performed according to methods known in the art.

### Pharmaceutical Compositions

In another aspect, the present disclosure provides use of pharmaceutical compositions comprising CCR5 binding agents described herein for administration to a patient in need thereof. Pharmaceutical compositions can comprise the CCR5 binding agents described herein and one or more pharmaceutically acceptable carriers, diluents, or excipients, suitable for administration by a selected route. A pharmaceutical composition can comprise any CCR5 binding agent described herein. Pharmaceutically acceptable carriers for diagnostic and therapeutic use are well known in the pharmaceutical art, and are described, for example, in Remington's Pharmaceutical Sciences, Mack Publishing Co. (A. R. Gennaro (Ed.), 18th Edition, 1990) and in CRC Handbook of Food, Drug, and Cosmetic Excipients, CRC Press LLC (S. C. Smolinski, ed., 1992). Exemplary pharmaceutically acceptable carriers include any adjuvant, carrier, excipient, glidant, diluent, preservative, dye/colorant, surfactant, wetting agent, dispersing agent, suspending agent, stabilizer, isotonic agent, solvent, emulsifier, or any combination thereof. For example, sterile saline and phosphate buffered saline at physiological pH can be suitable pharmaceutically acceptable carriers. Preservatives, stabilizers, dyes or the like may also be provided in the pharmaceutical composition. In addition, antioxidants and suspending agents may also be used. Pharmaceutical compositions may also contain diluents such as water, buffers, antioxidants such as ascorbic acid, low molecular weight polypeptides (less than about 10 residues), proteins, amino acids, carbohydrates (e.g., glucose, sucrose, dextrins), chelating agents (e.g., EDTA), glutathione, and other stabilizers and excipients. Neutral buffered saline or saline mixed with nonspecific serum albumin are exemplary diluents.

Pharmaceutical compositions comprising a CCR5 binding agent can be manufactured, for example, by lyophilizing the CCR5 binding agent, mixing, dissolving, emulsifying, encapsulating or entrapping the CCR5 binding agent. The pharmaceutical compositions can also include the CCR5 binding agents described herein in a free-base form or pharmaceutically-acceptable salt form.

A pharmaceutical composition may be formulated in the form of a solid, semi-solid or liquid composition. Solid compositions may include powders and tablets. In some embodiments, the pharmaceutical compositions described here are lyophilized or in powder form for re-constitution with a suitable vehicle, e.g., sterile water, before use. In some embodiments, the pharmaceutical compositions described herein is a suspension, solution, or emulsion. The pharmaceutical compositions and formulations can be sterilized. Sterilization can be accomplished by filtration through sterile filtration.

The pharmaceutical compositions described herein can be formulated for oral, topical, transdermal, inhalation, parenteral, sublingual, buccal, rectal, vaginal, and intranasal administration. The term “parenteral”, as used herein, includes subcutaneous, intravenous, intramuscular, intrasternal, and intratumoral injection or infusion techniques. In some embodiments, the pharmaceutical compositions described herein are formulated for administration as an injection, e.g., an intravenous or subcutaneous injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Alternatively, the pharmaceutical compositions described herein can be lyophilized or in powder form for reconstitution with a suitable vehicle, e.g., sterile pyrogen-free water, before use.

The CCR5 binding agents can be formulated for administration in a unit dosage form in association with a pharmaceutically acceptable vehicle. Such vehicles can be inherently nontoxic, and non-therapeutic. A vehicle can be water, saline, Ringer's solution, dextrose solution, and 5% human serum albumin. Non-aqueous vehicles such as fixed oils and ethyl oleate can also be used. The vehicle can contain minor amounts of additives such as substances that enhance isotonicity and chemical stability (e.g., buffers and preservatives).

In some embodiments, an aqueous formulation of a CCR5 binding agent provided herein, such as for subcutaneous administration, has a pH from 4-5.7. The aqueous formulation may comprise one or more excipients, such as, for example, one or more buffering agents, one or more lyoprotectants, and the like. In some embodiments, the pH of the formulation is from 4.0-6.0, 4.1-5.1, 4.2-5.1, 4.3-5.1, 4.4-5.1, 4.5-5.1, 4-5, 4.1-5, 4.2-5, 4.3-5, 4.4-5, 4.5-5, or about 4.5-5.5, about 5.3, about 5.4, about 5.5, about 5.6, or about 5.7. In some embodiments, the formulation comprises at least one buffer. In various embodiments, the buffer may be selected from histidine, citrate, aspartate, acetate, phosphate, lactate, tromethamine, gluconate, glutamate, tartrate, succinate, malic acid, fumarate, α-ketoglutarate, and combinations thereof. In some embodiments, the buffer is at least one buffer selected from histidine, citrate, aspartate, acetate, and combinations thereof. In some embodiments, the buffer is a combination of histidine and aspartate. In some embodiments, the total concentration of the buffer in the aqueous formulation is 10 mM to 40 mM, such as 15 mM-30 mM, 15 mM-25 mM, or 20 mM.

In some embodiments, the aqueous formulation comprises at least one lyoprotectant. In some such embodiments, the at least one lyoprotectant is selected from sucrose, arginine, glycine, sorbitol, glycerol, trehalose, dextrose, alpha-cyclodextrin, hydroxypropyl beta-cyclodextrin, hydroxypropyl gamma-cyclodextrin, proline, methionine, albumin, mannitol, maltose, dextran, and combinations thereof. In some embodiments, the lyoprotectant is sucrose. In some embodiments, the total concentration of lyoprotectant in the aqueous formulation is 3-12%, such as 5-12%, 6-10%, 5-9%, 7-9%, or 8%.

In some embodiments, the aqueous formulation comprises at least one surfactant. Exemplary surfactants include polysorbate 80, polysorbate 20, poloxamer 88, and combinations thereof. In some embodiments, the aqueous formulation comprises polysorbate 80. In some embodiments, the total concentration of the at least one surfactant is 0.01%-0.1%, such as 0.01%-0.05%, 0.01%-0.08%, or 0.01%-0.06%, 0.01%-0.04%, 0.01%-0.03%, or 0.02%.

In some embodiments, pharmaceutical compositions of the present invention are formulated in a single dose unit or in a form comprising a plurality of dosage units. Methods of preparing such dosage forms are known, or will be apparent, to those skilled in this art; for example, see Remington: The Science and Practice of Pharmacy, 20th Edition (Philadelphia College of Pharmacy and Science, 2000).

In some embodiments, the concentration of the CCR5 binding agent in the aqueous formulation is 1 mg/mL-250 mg/mL, such as 10 mg/mL-220 mg/mL, 10 mg/mL-200 mg/mL 10 mg/mL-175 mg/mL, 10 mg/mL-150 mg/mL, 10 mg/mL-100 mg/mL, 20 mg/mL-200 mg/mL, 20 mg/mL-175 mg/mL, 20 mg/mL-150 mg/mL, 20 mg/mL-125 mg/mL, 20 mg/mL-100 mg/mL, 30 mg/mL-200 mg/mL, 30 mg/mL-175 mg/mL, 30 mg/mL-150 mg/mL, 30 mg/mL-125 mg/mL, 30 mg/mL-100 mg/mL, 40 mg/mL-200 mg/mL 40 mg/mL-175 mg/mL, 40 mg/mL-150 mg/mL, 40 mg/mL-125 mg/mL, 40 mg/mL-100 mg/mL, 50 mg/mL-200 mg/mL 50 mg/mL-175 mg/mL, 50 mg/mL-150 mg/mL, 50 mg/mL-125 mg/mL, 50 mg/mL-100 mg/mL, 60 mg/mL-200 mg/mL, 60 mg/mL-175 mg/mL, 60 mg/mL-150 mg/mL, 60 mg/mL-125 mg/mL, 60 mg/mL-100 mg/mL, 70 mg/mL-200 mg/mL, 70 mg/mL-175 mg/mL, 70 mg/mL-150 mg/mL, 70 mg/mL-125 mg/mL, 80 mg/mL-200 mg/mL, 80 mg/mL-175 mg/mL, 80 mg/mL-150 mg/mL, 80 mg/mL-125 mg/mL, 100 mg/mL-200 mg/mL, 125 mg/mL-200 mg/mL, 150 mg/mL-200 mg/mL, or 160 mg/mL-190 mg/mL, 170 mg/mL-180 mg/mL, or 175 mg/mL. In some embodiments, the concentration of the CCR5 binding agent in the aqueous formulation is 100 mg/mL-200 mg/mL. In some embodiments, the concentration of the CCR5 binding agent in the aqueous formulation is 175 mg/mL.

In some embodiments, the CCR5 binding agent is an anti-CCR5 antibody or antigen binding fragment thereof formulated in a high protein concentration. High protein concentration formulations containing an exemplary anti-CCR5 antibody are described in U.S. Pat. No. 9,956,165 (incorporated by reference in its entirety).

In some embodiments, the anti-CCR5 antibody or antigen binding fragment is in a formulation comprising concentrated anti-CCR5 antibody or antigen binding fragment thereof in an amount greater than about 100 mg/mL and less than about 200 mg/mL; a tonicifier consisting essentially of a sodium salt and a histidine and glycine buffer present in a combined amount of from about 110 mM to about 120 mM and wherein the buffer is present in an amount of about 10 mM to about 25 mM; and a surfactant, wherein the formulation is hypotonic and has a total salt concentration of less than 100 mM.

In some embodiments, the anti-CCR5 antibody or antigen binding fragment is in a formulation comprising: concentrated anti-CCR5 antibody or antigen binding fragment in an amount greater than about 100 mg/mL and less than about 200 mg/mL; a sodium salt in an amount greater than about 90 mM and less than 100 mM; a histidine and glycine buffer in an amount greater than about 5 mM and less than about 25 mM; a surfactant in an amount greater than about 0.001% w/v and less than about 0.2% w/v; and, optionally, a stabilizing agent or non-salt tonicifier in an amount of about 0.05% w/v to about 1.8% w/v; wherein the formulation has an osmolality of about 250 to about 280 mOsm and has a total salt concentration of less than 100 mM.

In some embodiments, the anti-CCR5 antibody or antigen binding fragment is formulated in a low viscosity, hypotonic formulation, comprising: (a) concentrated anti-CCR5 antibody or antigen binding fragment in an amount greater than about 100 mg/mL and less than about 200 mg/mL; (b) a sodium salt in an amount selected from about 90 mM or about 95 mM; (c) a histidine and glycine buffer in an amount of about 20 mM; (d) a surfactant in an amount of 0.005% to 0.2% w/v; and optionally (e) a stabilizing agent or non-salt tonicifier in an amount sufficient to provide an osmolality of the formulation of about 260-280 mOs/kg; wherein the formulation has a total salt concentration of less than 100 mM.

In some embodiments, the anti-CCR5 antibody or antigen binding fragment is in a low viscosity hypotonic formulation, comprising: (a) concentrated anti-CCR5 antibody or antigen binding fragment in an amount greater than about 100 mg/mL and less than about 200 mg/mL; (b) a salt in an amount selected from about 90 mM or about 95 mM, wherein the salt is selected from sodium chloride, sodium gluconate, or sodium lactate; (c) a histidine and glycine buffer in an amount of about 20 mM; (d) a surfactant in an amount of about 0.005% to about 0.2% w/v, wherein the surfactant is a polysorbate, a poloxamer, or a pluronic; and (e) a stabilizing agent or non-salt tonicifier present in an amount sufficient to provide an osmolality of the formulation of about 230 mOs/kg to about 280 mOs/kg, wherein the stabilizing agent or non-salt tonicifier is selected from a sugar alcohol, a monosaccharide, a disaccharide, or a combination thereof; wherein the formulation has a total salt concentration of less than 100 mM.

In some embodiments, anti-CCR5 antibody or antigen binding fragment is formulated in a composition comprising anti-CCR5 antibody or antigen binding fragment in an amount greater than about 100 mg/mL and less than about 200 mg/mL, a tonicifier comprising a sodium salt present in a concentration of greater than about 90 mM and a histidine and glycine buffer present in a combined amount of from 110 mM to 120 mM and a surfactant present in an amount of from about 0.001% to about 0.2% w/v, wherein the composition has an osmolality of about 230 to about 290 mOs/kg and a total salt concentration of less than 100 mM.

In some embodiments, anti-CCR5 antibody or antigen binding fragment is provided as an article of manufacture comprising a container and a formulation comprising anti-CCR5 antibody or antigen binding fragment in a concentration of greater than 100 mg/mL and less than 200 mg/mL, a tonicifier of a sodium salt present in a concentration of greater than about 90 mM and a histidine and glycine buffer present in a combined amount of from about 110 mM to about 120 mM and the formulation has a total salt concentration of less than 100 mM, a surfactant in an amount of from about 0.005% to about 0.2%, and instructions for use.

In some embodiments, anti-CCR5 antibody or antigen binding fragment is administered in a dose of 700 mg of anti-CCR5 antibody or antigen binding fragment (175 mg/mL) delivered as two injections of 2 mL each and administered subcutaneously on opposite sides of the abdomen. Each vial of the anti-CCR5 antibody or antigen binding fragment product may contain ˜1.4 mL antibody at a concentration of 175 mg/mL.

In any of the aforementioned pharmaceutical compositions, the CCR5 binding agent may be leronlimab, a modified leronlimab, or a fragment thereof.

### Methods of Use

The present disclosure provides methods of treating a patient at risk of contracting HIV infection or a patient having an HIV infection by administering to the patient an effective amount of a CCR5 binding agent. In embodiments, the CCR5 binding agent comprises leronlimab or a binding fragment thereof. In further embodiments, the CCR5 binding agent comprises a leronlimab comprising one or more amino acid substitutions conferring increased effector function and circulation half-life. Results of in vivo studies in rhesus macaques demonstrated that a CCR5 binding agent with modifications to enhance effector function and circulation half-life can act as a PrEP agent for simian HIV infection.

Patients or patients that can be treated by CCR5 binding agents of the present disclosure include, but are not limited to, a mammal, such as human or non-human primates (e.g., monkeys and apes). In embodiments, the patient is human. The patient can be male or female and can be any suitable age, including infant, juvenile, adolescent, adult, and geriatric. In embodiments, the patient is >65 years. In some embodiments, the patient is ≤65 years. In embodiments, the patient is ≥18 years and ≤65 years.

Patients may also be administered a CCR5 binding agent in anticipation of exposure to HIV, upon exposure to HIV, at the initial detection of infection with HIV, upon the development of new symptoms after infection, or during any period of HIV infection, including AIDS.

Administration may be once or may continue for a set period of time, such as 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 6 months, 9 months, 1 year, 2 years, or until at least HIV viral RNA is no longer detectable.

Administration may be by weekly subcutaneous injection.

Due to the long-term availability of leronlimab after administration, less frequent administration, similar to that of other antibody therapeutics with Fc modifications, may be possible. For example, administration may be every two weeks, every month, every two months, every three months (12-weekly), every four months, every five months, or every six months. In specific embodiments administration may be every two months or every three months.

An appropriate dose, suitable duration, and frequency of administration of the CCR5 binding agents will be determined by such factors as the condition of the patient, size, weight, body surface area, age, sex, type and severity of the disease, particular therapy to be administered, particular form of the active ingredient, time and the method of administration, and other drugs being administered concurrently, which can readily be determined by a person skilled in the art.

Dosages can range from 0.1 to 100,000 μg/kg. Based upon the composition, the dose can be delivered continuously, such as by continuous pump, or at periodic intervals, e.g., on one or more separate occasions. Desired time intervals of multiple doses of a particular composition can be determined without undue experimentation by one skilled in the art.

In some embodiments, the CCR5 binding agent (e.g., leronlimab) is administered to the patient a plurality of times and each administration delivers from 0.01 mg per kg body weight to 50 mg per kg body weight of the antibody or binding fragment thereof to the patient. In another embodiment, each administration delivers from 0.05 mg per kg body weight to 25 mg per kg body weight of the CCR5 binding agent (e.g., leronlimab) to the patient. In a further embodiment, each administration delivers from 0.1 mg per kg body weight to 10 mg per kg body weight of the CCR5 binding agent (e.g., leronlimab) to the patient. In a still further embodiment, each administration delivers from 0.5 mg per kg body weight to 5 mg per kg body weight of the CCR5 binding agent (e.g., leronlimab) to the patient. In another embodiment, each administration delivers from 1 mg per kg body weight to 3 mg per kg body weight of the CCR5 binding agent (e.g., leronlimab) to the patient. In another embodiment, each administration delivers about 2 mg per kg body weight of the CCR5 binding agent (e.g., leronlimab) to the patient.

In one embodiment, the CCR5 binding agent (e.g., leronlimab) is administered once.

In one embodiment, the CCR5 binding agent (e.g., leronlimab) is administered twice.

In one embodiment, the CCR5 binding agent (e.g., leronlimab) is administered a plurality of times, and a first administration is separated from the subsequent administration by an interval of less than one week. In another embodiment, the first administration is separated from the subsequent administration by an interval of at least one week. In a further embodiment, the first administration is separated from the subsequent administration by an interval of one week. In another embodiment, the first administration is separated from the subsequent administration by an interval of two to four weeks. In another embodiment, the first administration is separated from the subsequent administration by an interval of two weeks. In a further embodiment, the first administration is separated from the subsequent administration by an interval of four weeks. In yet another embodiment, the CCR5 binding agent (e.g., leronlimab) is administered a plurality of times, and a first administration is separated from the subsequent administration by an interval of at least one month. In yet another embodiment, the CCR5 binding agent (e.g., leronlimab) is administered once a week for two weeks. In yet another embodiment, the CCR5 binding agent (e.g., leronlimab) is administered once a week for four weeks. In yet another embodiment, the CCR5 binding agent (e.g., leronlimab) is administered once per week as long as needed.

In a further embodiment, the CCR5 binding agent (e.g., leronlimab) is administered to the patient via intravenous infusion. In another embodiment, the CCR5 binding agent is administered to the patient via subcutaneous injection. In another embodiment, the CCR5 binding agent (e.g., leronlimab) is administered to the patient via intramuscular injection.

In some embodiments, the CCR5 binding agent (e.g., leronlimab) is administered at a once weekly dose of 350 mg to 1400 mg, or about 525 mg or about 700 mg or about 1050 mg. In some embodiments, the CCR5 binding agent (e.g., leronlimab) is administered at a twice weekly dose of 350 mg to 1400 mg, or about 525 mg or about 700 mg or about 1050 mg. In some embodiments, the CCR5 binding agent (e.g., leronlimab) is administered at a dose of about 700 mg, once weekly.

In some embodiments, the CCR5 binding agent (e.g. leronlimab) is administered at a once every two week, once monthly, once every two months, once every three months, once every four months, once every five months, or once every six months dose of 350 mg to 1400 mg, more specifically about 350 mg, about 525 mg, about 700 mg, or about 1050 mg.

In some embodiments, the CCR5 binding agent used in the methods described herein is leronlimab. Leronlimab (PRO 140) is currently under development for the indication of HIV, Graft versus host disease (GVHD), metastatic triple negative breast cancer (mTNBC), and metastatic colorectal cancer (mCRC). The safety profile of leronlimab (PRO 140) has been extensively evaluated in clinical trials. Leronlimab (PRO 140) has been administered intravenously or subcutaneously to more than 750 healthy and HIV-1 infected individuals in Phase I/II/III studies. The drug has been well tolerated following intravenous administration of single doses of 0.5 to 10 mg/kg or up to 700 mg weekly doses as subcutaneous (SC) injection. Overall, 324 patients have been exposed to leronlimab (PRO 140) 350 mg SC weekly dose with the longest duration of exposure lasting 4 years. Similarly, more than 250 and 150 patients have been exposed to leronlimab (PRO 140) 525 mg and 700 mg SC weekly dose, respectively.

In some embodiments, leronlimab is administered in a dose of 700 mg (175 mg/mL) delivered as two subcutaneous injections of 2 mL each on opposite sides of the abdomen.

### EXAMPLES

### Example 1: Pharmacokinetics of Leronlimab

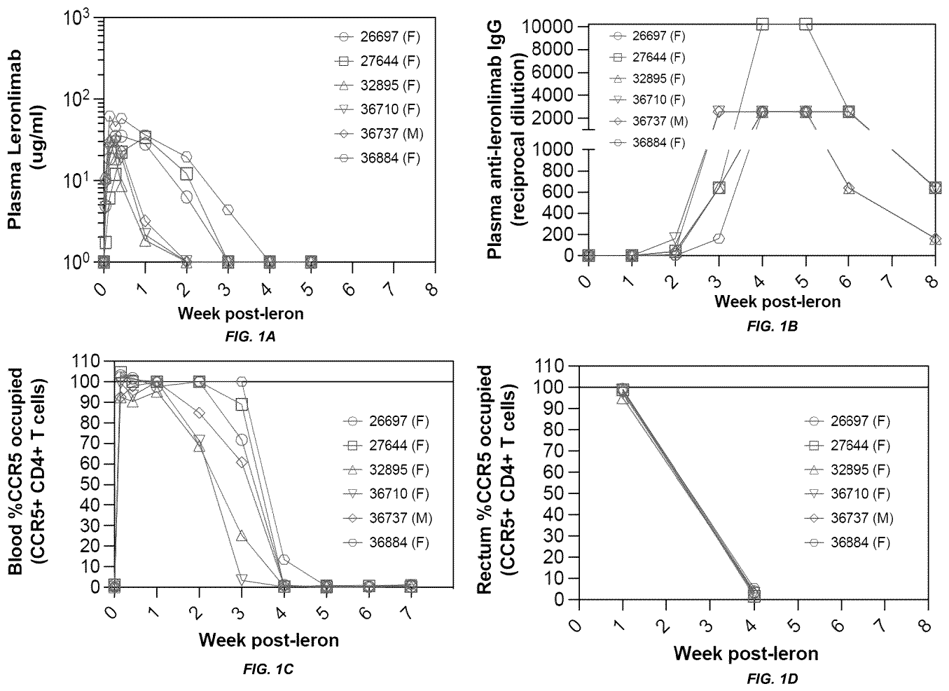

The current leronlimab formulation exhibits a plasma half-life of approximately ten days and surface CCR5 receptor occupancy (RO) lasting more than 60 days following a single dose. When a single 10 mg/kg dose of leronlimab was administered to macaques, transient levels of leronlimab in plasma were observed (FIG. 1A) and CCR5 RO on peripheral blood CD4+ T cells was rapidly cleared in all macaques by 4 or 5 weeks (FIG. 1C), respectively, post injection concomitant with the development of anti-drug antibodies (ADA) (FIG. 1B).

CCR5 RO on CD4+ T cells in the rectum at 4 weeks was also measured (FIG. 1D). Significantly, CCR5 RO at 4 weeks post-injection was non-detectable, indicating a long-term (at least 4 week) protective effect in rectal tissues.

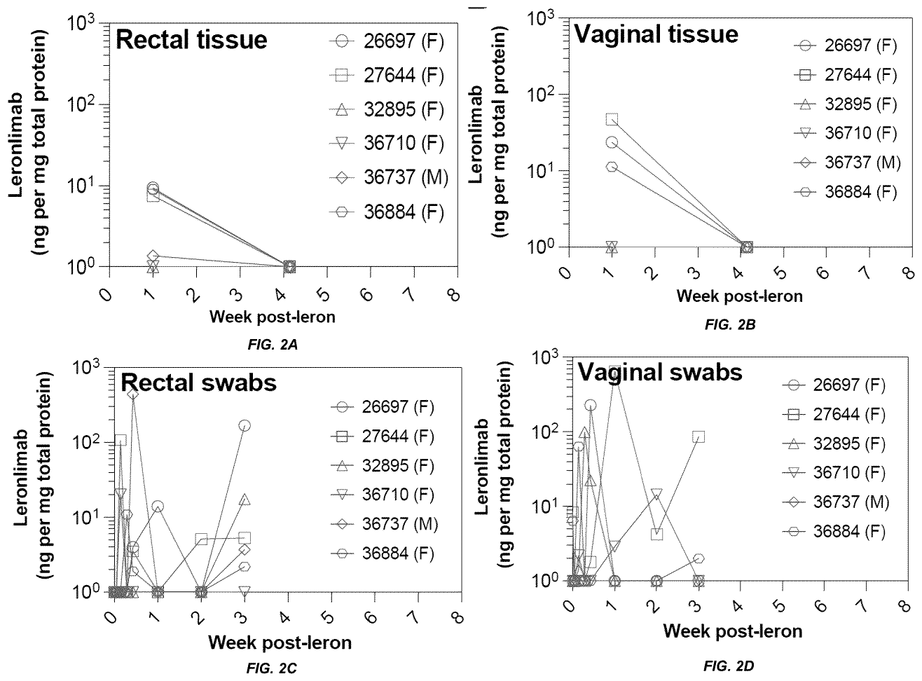

Leronlimab was present in both rectal and vaginal tissues (FIGS. 2A and 2B) and secretions (FIGS. 2C and 2D) at one week post injection, but absent at 4 weeks post infection.

Leronlimab was modified to extend half-life by increasing its interaction with the neonatal Fc receptor (FcRn), the major homeostatic regulator of antibody levels in plasma. To this end, leronlimab was modified as follows: 1) the antibody was “rhesus-ized” by replacing the human IgG4 Fc sequence with that of the corresponding macaque IgG4 Fc sequence, 2) M428L and N434S (“LS” mutation) mutations were inserted to increase serum half-life, 3) the double LALA (L234A and L235A) mutation was introduced to prevent engagement with Fc receptors, and 4) a S131C mutation was added to improve stability. The modified leronlimab is subsequently referred to as macaque IgG4 LS leronlimab and “MacLS”.

### Example 2: Pharmacokinetics of Macaque IgG4 LS Leronlimab

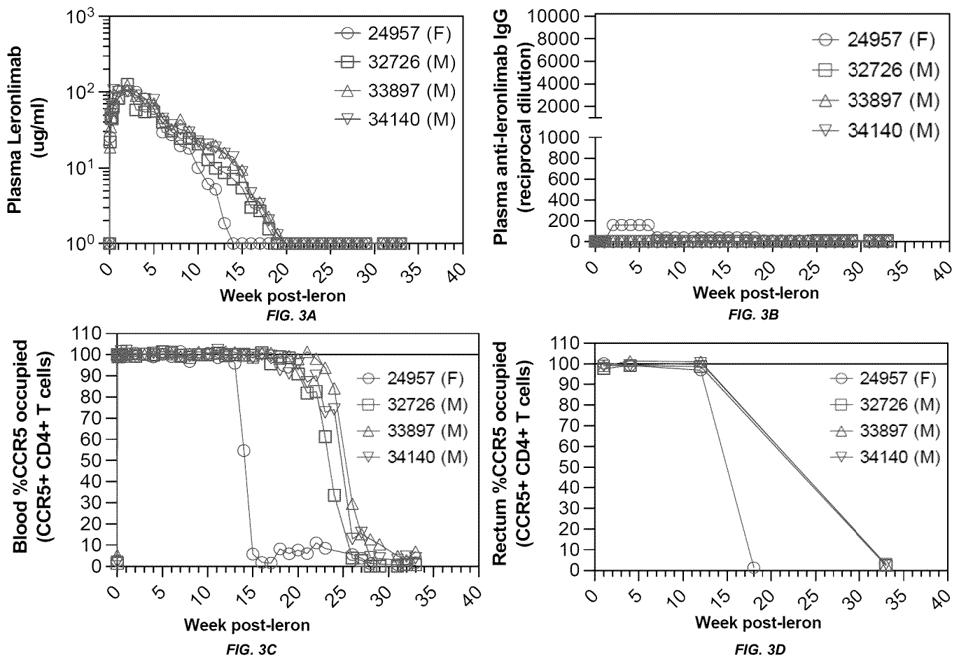

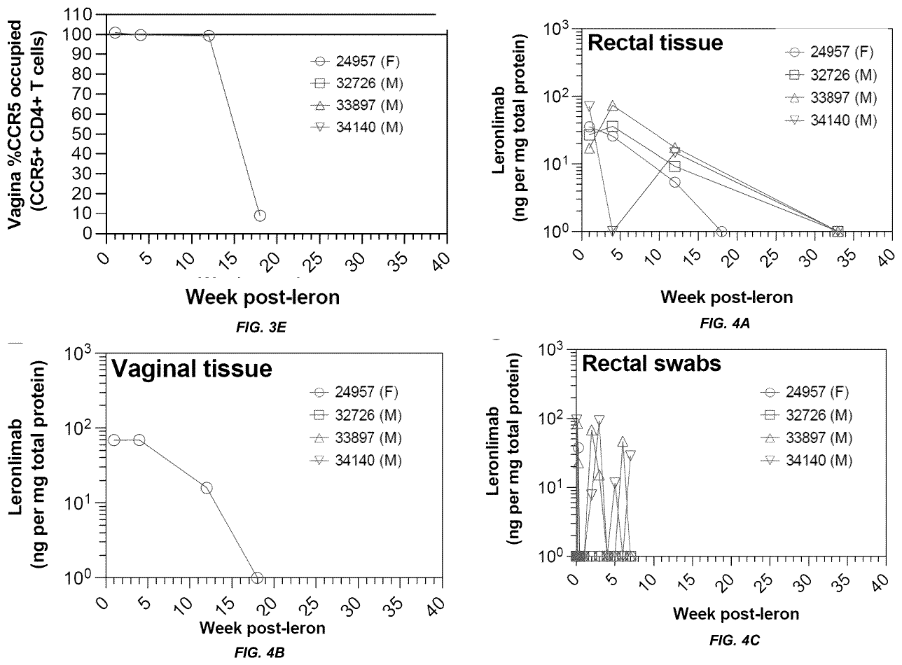

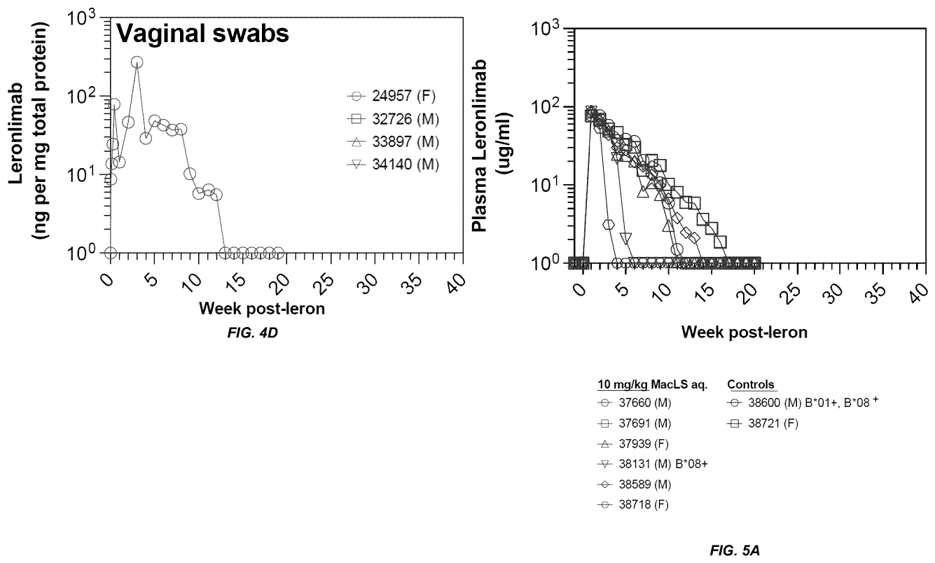

A 10 mg/kg dose of macaque IgG4 LS leronlimab (“MacLS”) was administered to four rhesus macaques. Leronlimab plasma levels (FIG. 3A) and CCR5 RO on blood CD4+ T cells (FIG. 3C) were elevated for significantly longer and ADA was not observed (FIG. 3B). CCR5 RO on CD4+ T cells in the rectum and vagina were also elevated for significantly longer (FIGS. 3D and 3E, respectively). The macaque IgG4 LS variant exhibited full CCR5 RO on peripheral blood CD4+ T cells for at least 13 weeks, with most macaques approaching 20 weeks of full CCR5 RO (FIG. 3C). In contrast, the parental leronlimab antibody only achieved full CCR5 RO for only 1-3 weeks on peripheral blood CD4+ T cells (FIG. 1C). Unexpectedly, the macaque IgG4 LS variant achieved full CCR5 RO on rectal and vaginal CD4+ T cells for at least 15 weeks (FIGS. 3D and 3E, respectively). This was likely due to much higher and sustained concentrations of this leronlimab variant in rectal and vaginal tissues and secretions (FIGS. 4A-4D).

### Example 3: In Vivo Pre-Exposure Prophylactic (PrEP) Activity of Macaque IgG4 LS Leronlimab

The pre-exposure prophylactic (PrEP) activity of macaque IgG4 LS leronlimab (“MacLS”) was tested in an in vivo rhesus macaque model of HIV infection. A single 10 mg/kg dose of MacLS was administered to six animals beginning at one week post-MacLS. Animals were challenged weekly at a low dose, intra-rectally with SHIV_{SF162P3}, which bears a HIV CCR5-tropic envelope. Two untreated rhesus macaques served as concurrent controls for the SHIV challenges.

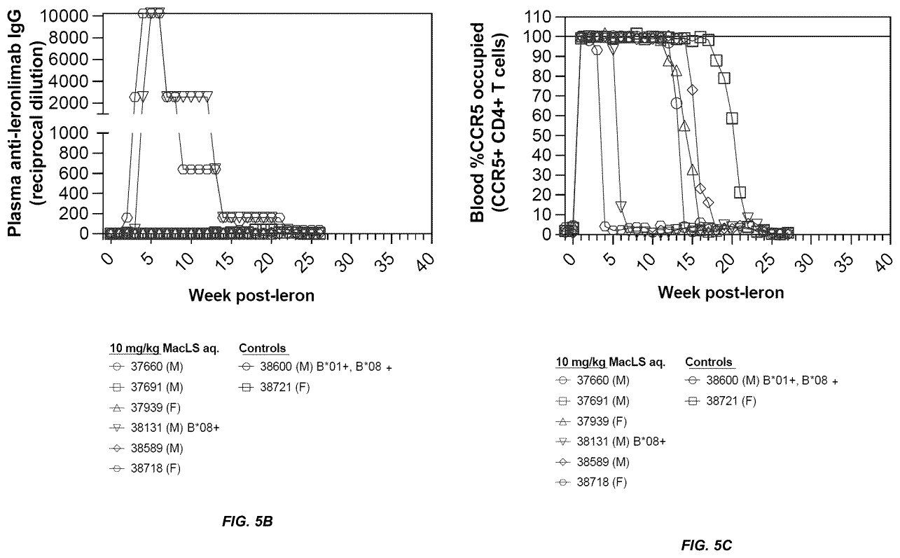

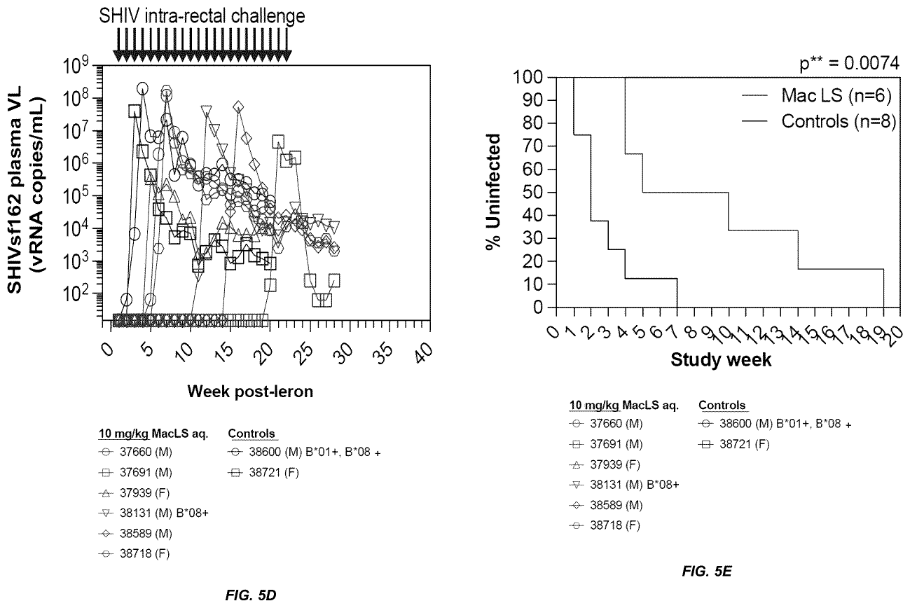

Elevated serum levels and full CCR5 RO on peripheral blood CD4+ T cells were observed for a longer period of time. Two macaques developed ADA (FIG. 5B), cleared leronlimab from plasma (FIG. 5A), and lost CCR5 RO (FIG. 5C). The single dose of macaque IgG4 LS variant significantly protected macaques from acquiring infection (FIGS. 5D and 5E) showing that a long-acting leronlimab molecule has the potential to act as a once-quarterly (once every 12 weeks) injectable for HIV prevention.

SEQUENCE LISTING

>SEQ ID NO: 1 VL protein sequence; signal peptide at amino acids 1-19; CDRs
underlined-
MKLPVRLLVLMFWIPASSSDIVMTQSPLSLPVTPGEPASISCRSSQRLLSSYGHTYLHWY
LQKPGQSPQLLIYEVSNRFSGVPDRFSGSGSGTDFTLKISRVEAEDVGVYYCSQSTHVPL
TFGQGTKVEIK
>SEQ ID NO: 2 VL nucleotide sequence
tctagaccaccatgaagttgcctgttaggctgttggtgctgatgttctggattcctgcttccagcagtgatattgtgatgacccaatctccactctc
cctgcctgtcactcctggagagccagcctccatctcttgcagatctagtcagcgccttctgagcagttatggacatacctatttacattggtacc
tacagaagccaggccagtctccacagctcctgatctacgaagtttccaaccgattttctggggtcccagacaggttcagtggcagtgggtca
gggacagatttcacacttaagatcagtagagtggaggctgaggatgtgggagtttattactgctctcaaagtacacatgttcctctcacgttcg
gacaggggaccaaggtggaaataaaacgtaagtagtcttctcaactctaga
>SEQ ID NO: 3 PRO#2 VH protein sequence; signal peptide at amino acids 1-19;
CDRs underlined
MEWSGVFIFLLSVTAGVHSEVQLVESGGGLVKPGGSLRLSCAASGYTFSNYWIGWVRQ
APGKGLEWIGDIYPGGNYIRNNEKFKDKTTLSADTSKNTAYLQMNSLKTEDTAVYYCG
SSFGSNYVFAWFTYWGQGTLVTVSS
>SEQ ID NO: 4 PRO#2 VH nucleotide sequence
acgcgtccaccatggaatggagcggagtctttatctttctcctgtcagtaactgcaggtgtccactccgaggtgcagctggtggagtctggtg
gaggcttggtaaagcctggaggttcccttagactctcctgtgcagcctctggttacactttcagtaactattggatcggatgggtccgccaggc
tccaggcaaagggctggagtggattggcgatatctaccctggagggaactacatcaggaacaatgagaagttcaaggacaagaccaccct
gtcagcagatacttccaagaacacagcctatctgcaaatgaacagcctgaaaaccgaggacacagccgtgtattactgtggaagcagcttc
ggtagtaactacgtgttcgcctggtttacttactggggccaagggactctggtcacagtctcctcaggtgagtccttaaaacctctaga
>SEQ ID NO: 5 PRO#1 VH protein sequence; signal peptide at amino acids 1-19;
CDRs underlined
MEWSGVFIFLLSVTAGVHSQVQLVQSGPDVKKPGTSMKMSCKTSGYTFSNYWIGWVR
QAPGQGLEWIGDIYPGGNYIRNNEKFKDKTTLTADTSTSTAYMQLGSLRSEDTAVYYCG
SSFGSNYVFAWFTYWGQGTLVTVSS
>SEQ ID NO: 6 PRO#1 VH nucleotide sequence
tctagaccaccatggaatggagcggggtctttatctttctcctgtcagtaactgcaggtgtccactcccaggtccaactggtgcagtctggacc
tgatgtgaaaaagcctgggacttcaatgaagatgtcctgcaagacgtctggatacaccttcagtaactattggatcggatgggttaggcaggc
gcctggacaaggccttgagtggattggagatatttaccctggagggaactatatcaggaacaatgagaagttcaaggacaagaccacactg
acggcagacacatcgaccagcacggcctacatgcaacttggcagcctgagatctgaagacactgccgtctattactgtggaagcagcttcg
gtagtaactacgtgttcgcctggtttacttactggggccaagggactctggtcacagtctcctcaggtgagtccttaaaacctctaga
>SEQ ID NO: 7 macaque IgG4 LS leronlimab heavy chain protein sequence-
CDRs underlined
EVQLVESGGGLVKPGGSLRLSCAASGYTFSNYWIGWVRQAPGKGLEWIGDIYPGGNYIR
NNEKFKDKTTLSADTSKNTAYLQMNSLKTEDTAVYYCGSSFGSNYVFAWFTYWGQGT
LVTVSSASTKGPSVFPLASCSRSTSESTAALGCLVKDYFPEPVTVSWNSGALTSGVHTFP
AVLQSSGLYSLSSVVTVPSSSLGTQTYVCNVVHEPSNTKVDKRVEFTPPCPPCPAPEAAG
GPSVFLFPPKPKDTLMISRTPEVTCVVVDVSQEDPEVQFNWYVDGVEVHNAQTKPRER
QFNSTYRVVSVLTVTHQDWLNGKEYTCKVSNKGLPAPIEKTISKAKGQPREPQVYILPPP
QEELTKNQVSLTCLVTGFYPSDIAVEWESNGQPENTYKTTPPVLDSDGSYLLYSKLTVN
KSRWQPGNIFTCSVLHEALHSHYTQKSLSVSPGK
>SEQ ID NO: 8 macaque IgG4 LS leronlimab light chain protein sequence-
CDRs underlined
DIVMTQSPLSLPVTPGEPASISCRSSQRLLSSYGHTYLHWYLQKPGQSPQLLIYEVSNRFS
GVPDRFSGSGSGTDFTLKISRVEAEDVGVYYCSQSTHVPLTFGQGTKVEIKRAVAAPSVF
IFPPSEDQVKSGTVSVVCLLNNFYPREASVKWKVDGVLKTGNSQESVTEQDSKDNTYSL
SSTLTLSSTDYQSHNVYACEVTHQGLSSPVTKSENRGEC
>SEQ ID NO: 9 LCDR1 amino acid sequence-RSSQRLLSSYGHTYLH
>SEQ ID NO: 10 LCDR2 amino acid sequence-EVSNRFS
>SEQ ID NO: 11 LCDR3 amino acid sequence-SQSTHVPLT
>SEQ ID NO: 12 HCDR1 amino acid sequence-NYWIG
>SEQ ID NO: 13 HCDR2 amino acid sequence-DIYPGGNYIRNNEKFKD
>SEQ ID NO: 14 HCDR3 amino acid sequence-SFGSNYVFAWFTY
>SEQ ID NO: 15 Homo sapiens CCR5 , NCBI Reference Sequence: NP_000570.1
MDYQVSSPIYDINYYTSEPCQKINVKQIAARLLPPLYSLVFIFGFVGNML VILILINCKRLK
SMTDIYLLNLAISDLFFLLTVPFWAHYAAAQWDFGNTMCQLLTGLYFIGFFSGIFFIILLTI
DRYLAVVHAVFALKARTVTFGVVTSVITWVVAVFASLPGIIFTRSQKEGLHYTCSSHFPY
SQYQFWKNFQTLKIVILGLVLPLLVMVICYSGILKTLLRCRNEKKRHRAVRLIFTIMIVYF
LFWAPYNIVLLLNTFQEFFGLNNCSSSNRLDQAMQVTETLGMTHCCINPIIYAFVGEKFR
NYLLVFFQKHIAKRFCKCCSIFQQEAPERASSVYTRSTGEQEISVGL
>SEQ ID NO: 16 [reserved]
>SEQ ID NO: 17 macaque IgG4 LS leronlimab heavy chain nucleotide sequence
GAGGTGCAGCTGGTGGAGTCTGGAGGAGGACTGGTGAAGCCAGGAGGAAGCCTGA
GGCTGTCTTGCGCCGCCTCCGGGTACACCTTCTCCAACTACTGGATCGGATGGGTGA
GACAGGCACCAGGCAAGGGACTGGAGTGGATCGGCGACATCTACCCCGGAGGCAA
CTACATCCGCAACAACGAGAAGTTCAAGGACAAGACCACCCTGAGCGCCGACACCT
CTAAGAACACCGCCTACCTGCAGATGAACTCTCTGAAGACCGAGGACACCGCCGTG
TACTACTGCGGATCCAGCTTCGGCTCCAACTACGTGTTCGCCTGGTTCACCTACTGG
GGACAGGGCACCCTGGTGACCGTGTCTTCCGCCAGCACCAAGGGACCATCCGTGTTC
CCACTGGCATCCTGCAGCAGGTCTACCTCCGAGAGCACCGCCGCCCTGGGATGCCTG
GTGAAGGACTACTTCCCTGAGCCAGTGACCGTGTCTTGGAACTCCGGGGCCCTGACC
TCCGGAGTGCACACCTTCCCAGCCGTGCTGCAGAGCTCTGGACTGTACAGCCTGTCC
AGCGTGGTGACCGTGCCTTCTTCCAGCCTGGGCACCCAGACCTACGTGTGCAACGTG
GTGCACGAGCCAAGCAACACCAAGGTGGACAAGAGGGTGGAGTTCACCCCACCTTG
CCCACCATGCCCAGCACCTGAGGCAGCAGGGGGACCCTCCGTGTTCCTGTTCCCTCC
AAAGCCTAAGGACACCCTGATGATCTCTCGCACCCCAGAGGTGACCTGCGTGGTGG
TGGACGTGTCCCAGGAGGACCCCGAGGTGCAGTTCAACTGGTACGTGGACGGGGTG
GAGGTGCACAACGCACAGACCAAGCCAAGGGAGCGCCAGTTCAACTCCACCTACAG
GGTGGTGAGCGTGCTGACCGTGACCCACCAGGACTGGCTGAACGGCAAGGAGTACA
CCTGCAAGGTGAGCAACAAGGGGCTGCCAGCCCCCATCGAGAAGACCATCTCTAAG
GCCAAGGGACAGCCCAGAGAGCCTCAGGTGTACATCCTGCCCCCTCCACAGGAGGA
GCTGACCAAGAACCAGGTGAGCCTGACCTGCCTGGTGACCGGCTTCTACCCTAGCG
ACATCGCCGTGGAGTGGGAGTCTAACGGGCAGCCAGAGAACACCTACAAGACCACC
CCACCTGTGCTGGACAGCGACGGGTCTTACCTGCTGTACTCCAAGCTGACCGTGAAC
AAGAGCAGGTGGCAGCCTGGAAACATCTTCACCTGCTCCGTGCTGCACGAGGCCCT
GCACAGCCACTACACTCAGAAGTCTCTGTCCGTGAGCCCAGGCAAG
>SEQ ID NO: 18 macaque IgG4 LS leronlimab light chain nucleotide sequence
GACATCGTGATGACCCAGTCCCCTCTGAGCCTGCCAGTGACCCCAGGAGAGCCTGCC
AGCATCTCTTGCAGGTCTTCCCAGAGACTGCTGAGCTCTTACGGACACACCTACCTG
CACTGGTACCTGCAGAAGCCTGGCCAGAGCCCACAGCTGCTGATCTACGAGGTGTC
CAACCGGTTCAGCGGAGTGCCAGACCGCTTCTCCGGAAGCGGGTCTGGAACCGACT
TCACCCTGAAGATCTCCAGGGTGGAGGCAGAGGACGTGGGCGTGTACTACTGCTCC
CAGAGCACCCACGTGCCACTGACCTTCGGCCAGGGGACCAAGGTGGAGATCAAGAG
GGCAGTGGCAGCACCTTCCGTGTTCATCTTCCCACCCTCCGAGGACCAGGTGAAGTC
TGGCACCGTGTCCGTGGTGTGCCTGCTGAACAACTTCTACCCCAGGGAGGCCTCCGT
GAAGTGGAAGGTGGACGGCGTGCTGAAGACCGGGAACTCTCAGGAGTCCGTGACCG
AGCAGGACTCTAAGGACAACACCTACTCCCTGTCCAGCACCCTGACCCTGTCTTCCA
CCGACTACCAGAGCCACAACGTGTACGCATGCGAGGTGACCCACCAGGGACTGAGC
TCTCCTGTGACCAAGTCTTTCAACAGAGGAGAGTGC
>SEQ ID NO: 19 macaque IgG4 LS leronlimab heavy chain protein sequence
with signal peptide (in bold)-CDRs underlined
MKHLWFFLLLVAAPRWVLSEVQLVESGGGLVKPGGSLRLSCAASGYTFSNYWIGWV
RQAPGKGLEWIGDIYPGGNYIRNNEKFKDKTTLSADTSKNTAYLQMNSLKTEDTAVYY
CGSSFGSNYVFAWFTYWGQGTLVTVSSASTKGPSVFPLASCSRSTSESTAALGCLVKDY
FPEPVTVSWNSGALTSGVHTFPAVLQSSGLYSLSSVVTVPSSSLGTQTYVCNVVHEPSNT
KVDKRVEFTPPCPPCPAPEAAGGPSVFLFPPKPKDTLMISRTPEVTCVVVDVSQEDPEVQ
FNWYVDGVEVHNAQTKPRERQFNSTYRVVSVLTVTHQDWLNGKEYTCKVSNKGLPAP
IEKTISKAKGQPREPQVYILPPPQEELTKNQVSLTCLVTGFYPSDIAVEWESNGQPENTYK
TTPPVLDSDGSYLLYSKLTVNKSRWQPGNIFTCSVLHEALHSHYTQKSLSVSPGK
>SEQ ID NO: 20 macaque IgG4 LS leronlimab light chain protein sequence
with signal peptide (in bold)-CDRs underlined
MKHLWFFLLLVAAPRWVLSDIVMTQSPLSLPVTPGEPASISCRSSQRLLSSYGHTYLH
WYLQKPGQSPQLLIYEVSNRFSGVPDRFSGSGSGTDFTLKISRVEAEDVGVYYCSQSTHV
PLTFGQGTKVEIKRAVAAPSVFIFPPSEDQVKSGTVSVVCLLNNFYPREASVKWKVDGV
LKTGNSQESVTEQDSKDNTYSLSSTLTLSSTDYQSHNVYACEVTHQGLSSPVTKSFNRG
EC
>SEQ ID NO: 21 macaque IgG4 LS leronlimab heavy chain nucleotide sequence
with signal peptide sequence (bold)
ATGAAGCACCTGTGGTTCTTCCTGCTGCTGGTGGCAGCACCAAGATGGGTGCT
GAGCGAGGTGCAGCTGGTGGAGTCTGGAGGAGGACTGGTGAAGCCAGGAGGAAGC
CTGAGGCTGTCTTGCGCCGCCTCCGGGTACACCTTCTCCAACTACTGGATCGGATGG
GTGAGACAGGCACCAGGCAAGGGACTGGAGTGGATCGGCGACATCTACCCCGGAG
GCAACTACATCCGCAACAACGAGAAGTTCAAGGACAAGACCACCCTGAGCGCCGAC
ACCTCTAAGAACACCGCCTACCTGCAGATGAACTCTCTGAAGACCGAGGACACCGC
CGTGTACTACTGCGGATCCAGCTTCGGCTCCAACTACGTGTTCGCCTGGTTCACCTA
CTGGGGACAGGGCACCCTGGTGACCGTGTCTTCCGCCAGCACCAAGGGACCATCCG
TGTTCCCACTGGCATCCTGCAGCAGGTCTACCTCCGAGAGCACCGCCGCCCTGGGAT
GCCTGGTGAAGGACTACTTCCCTGAGCCAGTGACCGTGTCTTGGAACTCCGGGGCCC
TGACCTCCGGAGTGCACACCTTCCCAGCCGTGCTGCAGAGCTCTGGACTGTACAGCC
TGTCCAGCGTGGTGACCGTGCCTTCTTCCAGCCTGGGCACCCAGACCTACGTGTGCA
ACGTGGTGCACGAGCCAAGCAACACCAAGGTGGACAAGAGGGTGGAGTTCACCCCA
CCTTGCCCACCATGCCCAGCACCTGAGGCAGCAGGGGGACCCTCCGTGTTCCTGTTC
CCTCCAAAGCCTAAGGACACCCTGATGATCTCTCGCACCCCAGAGGTGACCTGCGTG
GTGGTGGACGTGTCCCAGGAGGACCCCGAGGTGCAGTTCAACTGGTACGTGGACGG
GGTGGAGGTGCACAACGCACAGACCAAGCCAAGGGAGCGCCAGTTCAACTCCACCT
ACAGGGTGGTGAGCGTGCTGACCGTGACCCACCAGGACTGGCTGAACGGCAAGGAG
TACACCTGCAAGGTGAGCAACAAGGGGCTGCCAGCCCCCATCGAGAAGACCATCTC
TAAGGCCAAGGGACAGCCCAGAGAGCCTCAGGTGTACATCCTGCCCCCTCCACAGG
AGGAGCTGACCAAGAACCAGGTGAGCCTGACCTGCCTGGTGACCGGCTTCTACCCT
AGCGACATCGCCGTGGAGTGGGAGTCTAACGGGCAGCCAGAGAACACCTACAAGAC
CACCCCACCTGTGCTGGACAGCGACGGGTCTTACCTGCTGTACTCCAAGCTGACCGT
GAACAAGAGCAGGTGGCAGCCTGGAAACATCTTCACCTGCTCCGTGCTGCACGAGG
CCCTGCACAGCCACTACACTCAGAAGTCTCTGTCCGTGAGCCCAGGCAAG
>SEQ ID NO: 22 macaque IgG4 LS leronlimab light chain nucleotide sequence
with signal peptide sequence (bold)
ATGAAACACCTGTGGTTCTTCCTGTTACTCGTCGCCGCCCCAAGATGGGTGCTG
TCCGACATCGTGATGACCCAGTCCCCTCTGAGCCTGCCAGTGACCCCAGGAGAGCC
TGCCAGCATCTCTTGCAGGTCTTCCCAGAGACTGCTGAGCTCTTACGGACACACCTA
CCTGCACTGGTACCTGCAGAAGCCTGGCCAGAGCCCACAGCTGCTGATCTACGAGG
TGTCCAACCGGTTCAGCGGAGTGCCAGACCGCTTCTCCGGAAGCGGGTCTGGAACC
GACTTCACCCTGAAGATCTCCAGGGTGGAGGCAGAGGACGTGGGCGTGTACTACTG
CTCCCAGAGCACCCACGTGCCACTGACCTTCGGCCAGGGGACCAAGGTGGAGATCA
AGAGGGCAGTGGCAGCACCTTCCGTGTTCATCTTCCCACCCTCCGAGGACCAGGTGA
AGTCTGGCACCGTGTCCGTGGTGTGCCTGCTGAACAACTTCTACCCCAGGGAGGCCT
CCGTGAAGTGGAAGGTGGACGGCGTGCTGAAGACCGGGAACTCTCAGGAGTCCGTG
ACCGAGCAGGACTCTAAGGACAACACCTACTCCCTGTCCAGCACCCTGACCCTGTCT
TCCACCGACTACCAGAGCCACAACGTGTACGCATGCGAGGTGACCCACCAGGGACT
GAGCTCTCCTGTGACCAAGTCTTTCAACAGAGGAGAGTGC

1. A method of preventing HIV infection in a subject comprising administering to the subject an effective amount of a CCR5 antibody comprising the following human IgG Fc amino acid substitutions:
(i) M428L and N434S;
(ii) L234A and L235A; and
(iii) S131C
wherein the antibody comprises:
(a) a heavy chain variable region (VH) comprising a heavy chain complementary determining region 1 (HCDR1) of SEQ ID NO:11, a heavy chain complementary determining region 2 (HCDR2) of SEQ ID NO: 12, and a heavy chain complementary determining region 3 (HCDR3) of SEQ ID NO: 13; and
(b) a light chain variable region (VL) comprising a light chain complementary determining region 1 (LCDR1) of SEQ ID NO:9, a light chain complementary determining region 2 (LCDR2) of SEQ ID NO:10, and a light chain complementary determining region 3 (LCDR3) of SEQ ID NO:11.

2. The method of claim 1, wherein the CCR5 antibody is administered by injection.

3. The method of claim 1, wherein the CCR5 antibody is administered subcutaneously.

4. The method of claim 1, wherein the serum half-life of the CCR5 antibody is extended.

5. The method of claim 1, wherein the serum concentration of the CCR5 antibody is increased.

6. The method of claim 1, wherein the percentage of CCR5 receptors occupied by the CCR5 antibody on CCR5+ CD4+ T cells is increased.

7. The method of claim 1, wherein the period of time in which CCR5 receptors are occupied by the CCR5 antibody on CCR5+ CD4+ T cells is extended.

8. The method of claim 1, wherein the period of time in which CCR5 receptors are fully occupied by the CCR5 antibody on CCR5+ CD4+ T cells in the blood is at least 13 weeks.

9. The method of claim 1, wherein the period of time in which CCR5 receptors are fully occupied by the CCR5 antibody on CCR5+ CD4+ T cells in the rectum or vagina is at least 15 weeks.

10. The method of claim 1, wherein the CCR5 antibody is administered at a dose of about 350 mg.

11. The method of claim 1, wherein the CCR5 antibody is administered at a dose of about 525 mg.

12. The method of claim 1, wherein the CCR5 antibody is administered at a dose of about 700 mg.

13. The method of claim 1, wherein the CCR5 binding agent is administered at a dose of about 10 mg/kg.

14. The method of claim 1, wherein the CCR5 binding agent is administered at approximately 12-week intervals.

15. A method of conferring long-term CCR5 receptor occupancy (RO) in the vaginal or rectal tissue of a subject, the method comprising administering to the subject an effective amount of a CCR5 antibody comprising a human IgG Fc amino acid substitution, wherein the antibody comprises:
(a) a heavy chain variable region (VH) comprising a heavy chain complementary determining region 1 (HCDR1) of SEQ ID NO:11, a heavy chain complementary determining region 2 (HCDR2) of SEQ ID NO:12, and a heavy chain complementary determining region 3 (HCDR3) of SEQ ID NO:13; and
(b) a light chain variable region (VL) comprising a light chain complementary determining region 1 (LCDR1) of SEQ ID NO:9, a light chain complementary determining region 2 (LCDR2) of SEQ ID NO:10, and a light chain complementary determining region 3 (LCDR3) of SEQ ID NO:11,
wherein full receptor occupancy of CCR5 on CD4+ T cells in vaginal or rectal tissue of the subject persists for at least four weeks subsequent to administration.

16. The method of claim 15, wherein full receptor occupancy of CCR5 on CD4+ T cells in vaginal or rectal tissue of the subject persists for at least twelve weeks subsequent to administration.

17. The method of claim 15, wherein the human IgG Fc amino acid substitutions comprise M428L and N434S, L234A and L235A, and S131C.

18. A method of conferring long-term CCR5 receptor occupancy (RO) in the peripheral blood cells of a subject, the method comprising administering to the subject an effective amount of a CCR5 antibody comprising a human IgG Fc amino acid substitution, wherein the antibody comprises:
(a) a heavy chain variable region (VH) comprising a heavy chain complementary determining region 1 (HCDR1) of SEQ ID NO:11, a heavy chain complementary determining region 2 (HCDR2) of SEQ ID NO:12, and a heavy chain complementary determining region 3 (HCDR3) of SEQ ID NO:13; and
(b) a light chain variable region (VL) comprising a light chain complementary determining region 1 (LCDR1) of SEQ ID NO:9, a light chain complementary determining region 2 (LCDR2) of SEQ ID NO:10, and a light chain complementary determining region 3 (LCDR3) of SEQ ID NO:11,
wherein full receptor occupancy of CCR5 on CD4+ peripheral blood T cells of the subject persists for at least thirteen weeks subsequent to administration.

19. The method of claim 18, wherein full receptor occupancy of CCR5 on CD4+ T cells in vaginal or rectal tissue of the subject persists for at least twenty weeks subsequent to administration.

20. The method of claim 18, wherein the human IgG Fc amino acid substitutions comprise M428L and N434S, L234A and L235A, and S131C.


SEQUENCE LISTING (22 sequences)

SEQ ID NO: 1 - protein, 131 aa, synthetic construct
MKLPVRLLVLMFWIPASSSDIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIK

SEQ ID NO: 2 - DNA, 429 bp, synthetic construct
  source 1..429: mol_type = other DNA
tctagaccaccatgaagttgcctgttaggctgttggtgctgatgttctggattcctgcttccagcagtgatattgtgatgacccaatctccactctccctgcctgtcactcctggagagccagcctccatctcttgcagatctagtcagcgccttctgagcagttatggacatacctatttacattggtacctacagaagccaggccagtctccacagctcctgatctacgaagtttccaaccgattttctggggtcccagacaggttcagtggcagtgggtcagggacagatttcacacttaagatcagtagagtggaggctgaggatgtgggagtttattactgctctcaaagtacacatgttcctctcacgttcggacaggggaccaaggtggaaataaaacgtaagtagtcttctcaactctaga

SEQ ID NO: 3 - protein, 141 aa, synthetic construct
MEWSGVFIFLLSVTAGVHSEVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSS

SEQ ID NO: 4 - DNA, 457 bp, synthetic construct
  source 1..457: mol_type = other DNA
acgcgtccaccatggaatggagcggagtctttatctttctcctgtcagtaactgcaggtgtccactccgaggtgcagctggtggagtctggtggaggcttggtaaagcctggaggttcccttagactctcctgtgcagcctctggttacactttcagtaactattggatcggatgggtccgccaggctccaggcaaagggctggagtggattggcgatatctaccctggagggaactacatcaggaacaatgagaagttcaaggacaagaccaccctgtcagcagatacttccaagaacacagcctatctgcaaatgaacagcctgaaaaccgaggacacagccgtgtattactgtggaagcagcttcggtagtaactacgtgttcgcctggtttacttactggggccaagggactctggtcacagtctcctcaggtgagtccttaaaacctctaga

SEQ ID NO: 5 - protein, 141 aa, synthetic construct
MEWSGVFIFLLSVTAGVHSQVQLVQSGPDVKKPGTSMKMSCKTSGYTFSNYWIGWVRQAPGQGLEWIGDIYPGGNYIRNNEKFKDKTTLTADTSTSTAYMQLGSLRSEDTAVYYCGSSFGSNYVFAWFTYWGQGTLVTVSS

SEQ ID NO: 6 - DNA, 457 bp, synthetic construct
  source 1..457: mol_type = other DNA
tctagaccaccatggaatggagcggggtctttatctttctcctgtcagtaactgcaggtgtccactcccaggtccaactggtgcagtctggacctgatgtgaaaaagcctgggacttcaatgaagatgtcctgcaagacgtctggatacaccttcagtaactattggatcggatgggttaggcaggcgcctggacaaggccttgagtggattggagatatttaccctggagggaactatatcaggaacaatgagaagttcaaggacaagaccacactgacggcagacacatcgaccagcacggcctacatgcaacttggcagcctgagatctgaagacactgccgtctattactgtggaagcagcttcggtagtaactacgtgttcgcctggtttacttactggggccaagggactctggtcacagtctcctcaggtgagtccttaaaacctctaga

SEQ ID NO: 7 - protein, 447 aa, synthetic construct
EVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSASTKGPSVFPLASCSRSTSESTAALGCLVKDYFPEPVTVSWNSGALTSGVHTFPAVLQSSGLYSLSSVVTVPSSSLGTQTYVCNVVHEPSNTKVDKRVEFTPPCPPCPAPEAAGGPSVFLFPPKPKDTLMISRTPEVTCVVVDVSQEDPEVQFNWYVDGVEVHNAQTKPRERQFNSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYLLYSKLTVNKSRWQPGNIFTCSVLHEALHSHYTQKSLSVSPGK

SEQ ID NO: 8 - protein, 219 aa, synthetic construct
DIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKRAVAAPSVFIFPPSEDQVKSGTVSVVCLLNNFYPREASVKWKVDGVLKTGNSQESVTEQDSKDNTYSLSSTLTLSSTDYQSHNVYACEVTHQGLSSPVTKSFNRGEC

SEQ ID NO: 9 - protein, 16 aa, synthetic construct
RSSQRLLSSYGHTYLH

SEQ ID NO: 10 - protein, 7 aa, synthetic construct
EVSNRFS

SEQ ID NO: 11 - protein, 9 aa, synthetic construct
SQSTHVPLT

SEQ ID NO: 12 - protein, 5 aa, synthetic construct
NYWIG

SEQ ID NO: 13 - protein, 17 aa, synthetic construct
DIYPGGNYIRNNEKFKD

SEQ ID NO: 14 - protein, 13 aa, synthetic construct
SFGSNYVFAWFTY

SEQ ID NO: 15 - protein, 352 aa, Homo sapiens [sequence omitted]

SEQ ID NO: 16 - skipped

SEQ ID NO: 17 - DNA, 1341 bp, synthetic construct
  source 1..1341: mol_type = other DNA
gaggtgcagctggtggagtctggaggaggactggtgaagccaggaggaagcctgaggctgtcttgcgccgcctccgggtacaccttctccaactactggatcggatgggtgagacaggcaccaggcaagggactggagtggatcggcgacatctaccccggaggcaactacatccgcaacaacgagaagttcaaggacaagaccaccctgagcgccgacacctctaagaacaccgcctacctgcagatgaactctctgaagaccgaggacaccgccgtgtactactgcggatccagcttcggctccaactacgtgttcgcctggttcacctactggggacagggcaccctggtgaccgtgtcttccgccagcaccaagggaccatccgtgttcccactggcatcctgcagcaggtctacctccgagagcaccgccgccctgggatgcctggtgaaggactacttccctgagccagtgaccgtgtcttggaactccggggccctgacctccggagtgcacaccttcccagccgtgctgcagagctctggactgtacagcctgtccagcgtggtgaccgtgccttcttccagcctgggcacccagacctacgtgtgcaacgtggtgcacgagccaagcaacaccaaggtggacaagagggtggagttcaccccaccttgcccaccatgcccagcacctgaggcagcagggggaccctccgtgttcctgttccctccaaagcctaaggacaccctgatgatctctcgcaccccagaggtgacctgcgtggtggtggacgtgtcccaggaggaccccgaggtgcagttcaactggtacgtggacggggtggaggtgcacaacgcacagaccaagccaagggagcgccagttcaactccacctacagggtggtgagcgtgctgaccgtgacccaccaggactggctgaacggcaaggagtacacctgcaaggtgagcaacaaggggctgccagcccccatcgagaagaccatctctaaggccaagggacagcccagagagcctcaggtgtacatcctgccccctccacaggaggagctgaccaagaaccaggtgagcctgacctgcctggtgaccggcttctaccctagcgacatcgccgtggagtgggagtctaacgggcagccagagaacacctacaagaccaccccacctgtgctggacagcgacgggtcttacctgctgtactccaagctgaccgtgaacaagagcaggtggcagcctggaaacatcttcacctgctccgtgctgcacgaggccctgcacagccactacactcagaagtctctgtccgtgagcccaggcaag

SEQ ID NO: 18 - DNA, 657 bp, synthetic construct
  source 1..657: mol_type = other DNA
gacatcgtgatgacccagtcccctctgagcctgccagtgaccccaggagagcctgccagcatctcttgcaggtcttcccagagactgctgagctcttacggacacacctacctgcactggtacctgcagaagcctggccagagcccacagctgctgatctacgaggtgtccaaccggttcagcggagtgccagaccgcttctccggaagcgggtctggaaccgacttcaccctgaagatctccagggtggaggcagaggacgtgggcgtgtactactgctcccagagcacccacgtgccactgaccttcggccaggggaccaaggtggagatcaagagggcagtggcagcaccttccgtgttcatcttcccaccctccgaggaccaggtgaagtctggcaccgtgtccgtggtgtgcctgctgaacaacttctaccccagggaggcctccgtgaagtggaaggtggacggcgtgctgaagaccgggaactctcaggagtccgtgaccgagcaggactctaaggacaacacctactccctgtccagcaccctgaccctgtcttccaccgactaccagagccacaacgtgtacgcatgcgaggtgacccaccagggactgagctctcctgtgaccaagtctttcaacagaggagagtgc

SEQ ID NO: 19 - protein, 466 aa, synthetic construct
MKHLWFFLLLVAAPRWVLSEVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSASTKGPSVFPLASCSRSTSESTAALGCLVKDYFPEPVTVSWNSGALTSGVHTFPAVLQSSGLYSLSSVVTVPSSSLGTQTYVCNVVHEPSNTKVDKRVEFTPPCPPCPAPEAAGGPSVFLFPPKPKDTLMISRTPEVTCVVVDVSQEDPEVQFNWYVDGVEVHNAQTKPRERQFNSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYLLYSKLTVNKSRWQPGNIFTCSVLHEALHSHYTQKSLSVSPGK

SEQ ID NO: 20 - protein, 238 aa, synthetic construct
MKHLWFFLLLVAAPRWVLSDIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKRAVAAPSVFIFPPSEDQVKSGTVSVVCLLNNFYPREASVKWKVDGVLKTGNSQESVTEQDSKDNTYSLSSTLTLSSTDYQSHNVYACEVTHQGLSSPVTKSFNRGEC

SEQ ID NO: 21 - DNA, 1398 bp, synthetic construct
  source 1..1398: mol_type = other DNA
atgaagcacctgtggttcttcctgctgctggtggcagcaccaagatgggtgctgagcgaggtgcagctggtggagtctggaggaggactggtgaagccaggaggaagcctgaggctgtcttgcgccgcctccgggtacaccttctccaactactggatcggatgggtgagacaggcaccaggcaagggactggagtggatcggcgacatctaccccggaggcaactacatccgcaacaacgagaagttcaaggacaagaccaccctgagcgccgacacctctaagaacaccgcctacctgcagatgaactctctgaagaccgaggacaccgccgtgtactactgcggatccagcttcggctccaactacgtgttcgcctggttcacctactggggacagggcaccctggtgaccgtgtcttccgccagcaccaagggaccatccgtgttcccactggcatcctgcagcaggtctacctccgagagcaccgccgccctgggatgcctggtgaaggactacttccctgagccagtgaccgtgtcttggaactccggggccctgacctccggagtgcacaccttcccagccgtgctgcagagctctggactgtacagcctgtccagcgtggtgaccgtgccttcttccagcctgggcacccagacctacgtgtgcaacgtggtgcacgagccaagcaacaccaaggtggacaagagggtggagttcaccccaccttgcccaccatgcccagcacctgaggcagcagggggaccctccgtgttcctgttccctccaaagcctaaggacaccctgatgatctctcgcaccccagaggtgacctgcgtggtggtggacgtgtcccaggaggaccccgaggtgcagttcaactggtacgtggacggggtggaggtgcacaacgcacagaccaagccaagggagcgccagttcaactccacctacagggtggtgagcgtgctgaccgtgacccaccaggactggctgaacggcaaggagtacacctgcaaggtgagcaacaaggggctgccagcccccatcgagaagaccatctctaaggccaagggacagcccagagagcctcaggtgtacatcctgccccctccacaggaggagctgaccaagaaccaggtgagcctgacctgcctggtgaccggcttctaccctagcgacatcgccgtggagtgggagtctaacgggcagccagagaacacctacaagaccaccccacctgtgctggacagcgacgggtcttacctgctgtactccaagctgaccgtgaacaagagcaggtggcagcctggaaacatcttcacctgctccgtgctgcacgaggccctgcacagccactacactcagaagtctctgtccgtgagcccaggcaag

SEQ ID NO: 22 - DNA, 714 bp, synthetic construct
  source 1..714: mol_type = other DNA
atgaaacacctgtggttcttcctgttactcgtcgccgccccaagatgggtgctgtccgacatcgtgatgacccagtcccctctgagcctgccagtgaccccaggagagcctgccagcatctcttgcaggtcttcccagagactgctgagctcttacggacacacctacctgcactggtacctgcagaagcctggccagagcccacagctgctgatctacgaggtgtccaaccggttcagcggagtgccagaccgcttctccggaagcgggtctggaaccgacttcaccctgaagatctccagggtggaggcagaggacgtgggcgtgtactactgctcccagagcacccacgtgccactgaccttcggccaggggaccaaggtggagatcaagagggcagtggcagcaccttccgtgttcatcttcccaccctccgaggaccaggtgaagtctggcaccgtgtccgtggtgtgcctgctgaacaacttctaccccagggaggcctccgtgaagtggaaggtggacggcgtgctgaagaccgggaactctcaggagtccgtgaccgagcaggactctaaggacaacacctactccctgtccagcaccctgaccctgtcttccaccgactaccagagccacaacgtgtacgcatgcgaggtgacccaccagggactgagctctcctgtgaccaagtctttcaacagaggagagtgc